(19) United States
(12) Patent Application Publication
(10) Pub. No.: US 2008/0154212 A1
(43) Pub. Date: Jun. 26, 2008

(54) SYRINGE WITH RETRACTABLE NEEDLE SUPPORT

(75) Inventor: Steven SCHRAGA, Surfside, FL (US)

Correspondence Address: GREENBLUM & BERNSTEIN, P.L.C., 1950 ROLAND CLARKE PLACE, RESTON, VA, 20191, US

(73) Assignee: STAT MEDICAL DEVICES, INC., North Miami, FL (US)

(21) Appl. No.: 11/616,196

(22) Filed: Dec. 26, 2006

(51) Int. Cl. A61M 5/315

(52) U.S. Cl. 604/220

(57) ABSTRACT

An injection device includes a barrel, a plunger having a portion structured and arranged to move within the barrel, a needle support, and a locking arrangement. The locking arrangement may at least one of selectively lock a proximal end portion of the plunger to the needle support and automatically lock a proximal end portion of the plunger to an outer portion of the needle support. This Abstract is not intended to define the invention disclosed in the specification, nor intended to limit the scope of the invention in any way.

### BACKGROUND OF THE INVENTION

1. Field of the Invention

This invention relates generally to syringes, e.g., hypodermic syringes, such are utilized for injection of medicament into the body tissues of human and animal patients. More specifically, this invention relates to a hypodermic syringe having a plunger, piston and needle support structure that permits retraction of the needle support and its needle into the barrel of the syringe to prevent the possibility of inadvertent needle pricks and which incorporates a frangible plunger that may be broken away to prevent subsequent actuation of the needle to its operative position.

This invention also relates to syringes which have minimal dead-space so that they can be ideally used for injecting very expensive medicaments with minimal waste.

This invention also relates to syringes which can be used only once, i.e., single-use syringes, and/or to syringes which utilize a rotating releasable lock system for locking the plunger to the needle support when desired.

2. Discussion of Background Information

In hospitals, nursing home facilities and the like, injection of medicament into the body tissues of patients is done on a daily basis. Typical hypodermic syringes are provided with a barrel having a needle that is fixed or removably attached at one end thereof. A plunger typically having an elastomeric piston is movable within the barrel to load the barrel with liquid medicament by suction as the plunger and piston are moved within the barrel in a direction away from the needle. After the needle has penetrated the body tissues of the patient, as the direction of movement of the plunger and piston are reversed and the piston is forced toward the needle, medicament contained within the barrel will be injected through the needle into the body tissues.

After hypodermic syringes have been used in this manner, those syringes that are disposable present a significant problem to users, e.g., hospital or nursing home staff, because the possibility of inadvertent needle pricks subject personnel to the possibility of cross-contamination by, among other things, virile or bacterial contaminants that might be present on the needle after its use. In an effort to avoid the possibility of inadvertent needle pricks special waste containers are often provided at hospital facilities into which the used disposable hypodermic syringes are placed. These containers and the syringes contained therein are then disposed of in a specifically organized manner to insure against the possibility of inadvertent infectious contamination of nursing personnel. Further, refuse handlers and other persons who might inadvertently come into contact with the used hypodermic syringes are also subject to the same hazards. Often times the needles themselves are bent over so as to minimize the possibility of inadvertent needle pricks and to preclude the possibility of subsequent use of disposable hypodermic syringes.

In certain situations, medicaments are injected into patients and not quickly thereafter discarded properly. Instead, the used syringe is placed in a temporary position. After the procedure has ended, the syringe can be manually recovered for disposal. However, between the time of use and the time of disposal, there is the possibility that inadvertent needle pricks will occur. Accordingly, it is desirable to provide a suitable way protecting personnel, e.g., nursing personnel, paramedics and other persons, from the hazards of inadvertent needle pricks as they go about their daily tasks.

U.S. Pat. Nos. 5,328,475, 5,591,131, and 5,569,203 to CHEN, the disclosures of which are hereby expressly incorporated by reference in their entireties, discloses a hypodermic syringe which locks the plunger to the needle support when the plunger is moved to the full depth position during injection. During injection, however, the plunger irreversibly locks to the needle support when the plunger is moved to the full depth position. Furthermore, the plunger cannot be moved to the full depth position without causing engagement of the lock system. This means that there will be a dead space in the syringe if the plunger is not moved to the full depth position. This type of syringe is shown in FIGS. 1-3 wherein the syringe barrel 1 has an internal cylindrical space for housing the medicine, a plunger 2 having an elastomer sealing piston is slidable engaged in the barrel 1 so as to cause the medicine to enter and exit the barrel 1, and a needle support 3 is arranged in sealing engagement with a front end opening of the syringe. After the syringe is used for injecting a medicine, the plunger 2 is moved to the full injection position wherein it becomes automatically locked to the needle support 3. Once the plunger 2 becomes locked to the needle support 3, the plunger 2 can be used to fully retract the needle support 3 into barrel 1. The rear end of the plunger 2 can then be snapped-off so as to prevent reuse of the syringe and ensure that the syringe cannot inadvertently prick the skin of anyone handling the syringe.

U.S. Pat. No. 5,098,402 to DAVIS, the disclosure of which is hereby expressly incorporated by reference in its entirety, discloses a hypodermic syringe which locks the plunger to the needle support when the plunger is moved to the full depth position during injection. However, during injection, the plunger irreversibly locks to the needle support when the plunger is moved to the full depth position. Furthermore, the plunger cannot be moved to the full depth position without causing engagement of the lock system. This means that there will be a dead space in the syringe if the plunger is not moved to the full depth position. Moreover, the disclosed lock system prevents the plunger from rotating relative to needle support due to engagement between the plunger and the needle support. Finally, the plunger must be rotated to cause the needle support to rotate from a locked position to a position which allows the needle support to be retracted into the syringe barrel.

U.S. Pat. No. 6,461,328 to WANG et al., the disclosure of which is hereby expressly incorporated by reference in its entirety, discloses a hypodermic syringe which locks the plunger to the needle support hub when the plunger is moved to the full depth position during injection. However, during injection, the plunger irreversibly locks to the needle support hub when the plunger is moved to the full depth position. Furthermore, the plunger cannot be moved to the full depth position without causing engagement of the lock system. This means that there will be a dead space in the syringe if the plunger is not moved to the full depth position. Moreover, the disclosed lock system prevents the plunger from rotating relative to needle support hub due to engagement between the plunger and the needle support hub. Finally, the plunger must be rotated to cause the needle support hub and the needle support secured thereto to rotate from a locked position to a position which allows the needle support hub and needle support to be retracted into the syringe barrel.

it is therefore desirable to provide a syringe that includes a system for rendering the needle thereof to a protected, completely encapsulated condition such that it is less likely to cause, after use, an inadvertent needle prick during its handling or during its disposal. It is also desirable to provide a syringe having the capability of retracting the needle to a position inside the barrel of the syringe and locking the needle in its retracted position so that the needle of the syringe is always enclosed after its use, thus precluding the possibility that the needle might cause an accidental needle prick as the syringe is subsequently handled. It is also desirable to provide a syringe of the disposable type that is provided with facility for rendering it completely inoperative such that it can not be subsequently used. Additionally, it is desirable to provide for a syringe which also has minimal dead-space so that it can be ideally used for injecting very expensive medicaments with minimal waste. Finally, it is desirable to provide for a syringe which also has a system for selectively locking the plunger to the needle support so that the syringe can have minimal dead-space and so that the plunger and be locked with the needle support only when desired.

### SUMMARY OF THE INVENTION

According to one non-limiting aspect of the invention there is provided a hypodermic syringe having a plunger which can, in one orientation, be moved to the full depth position so as to allow essentially all of the contents of the syringe to be injected without causing the plunger to become locked with the needle support or the needle support hub and, in another orientation, a plunger can be moved to the full depth position so as to allow essentially all of the contents of the syringe to be injected while also causing the plunger to become locked with the needle support or the needle support hub. If the user desires, the plunger, while in the full depth position, can be rotated to cause unlocking of the plunger and needle support or can be rotated to cause locking of the plunger and needle support.

According to one non-limiting aspect of the invention there is provided a prior art hypodermic syringe, e.g., of the type discussed above, that is modified to include one or more features of the invention described herein. By way of non-limiting example, the needle support or needle support hub can be press fit in a simple manner and with a simple design into the end opening of the syringe barrel and need not require the complex connection arrangements of the prior art discussed above.

The invention also provides for a syringe whose needle support and/or needle support hub is capable of being easily and simply retracted to a safe position within the barrel where the needle is withdrawn to an enclosed and protected position preventing the possibility of inadvertent needle pricks.

The invention also provides for a retractable hypodermic syringe having a frangible plunger that may be manually broken after the syringe has been used and the needle has been retracted and locked safely within the syringe barrel to thus render the syringe inoperative for further use.

The invention also provides for a syringe including an internal selective locking system that utilizes a mechanical interaction between with a portion of the plunger to achieve locking of the needle support and/or needle support hub so as to allow the plunger to retract the needle support and/or needle support hub into the barrel of the syringe such that the needle is rendered incapable of causing an inadvertent needle prick.

The invention is also directed to a retractable hypodermic syringe that includes one or more of the following features: a syringe body or cylindrical barrel. The barrel is designed to received therein a plunger. The proximal end of the plunger includes an elastomeric piston that sealingly engages with the internal cylindrical surface of the barrel. The distal end of the syringe body defines an opening which is sized and configured to receive therein a needle support. The needle support has a hypodermic needle affixed thereto, is press-fit (so as to be in sealingly engagement with) into the proximal opening of the barrel, and is also capable of being withdrawn into the barrel. The needle support is therefore capable of establishing a sealing engagement internally of the syringe body and defines a receptacle within which is received a connecting and actuating projection extending from the plunger such that a rotatable locking/unlocking relationship is established between the plunger and the needle support. Linear movement of the plunger in a direction away from the needle support is adapted to cause retracting movement of the needle support and needle to thus retract the needle to a protected position within the syringe barrel. The plunger and syringe barrel are also adapted for locking a portion of the plunger, the piston and needle support in a safe retracted position within the syringe barrel and for breaking away the operating portion of the plunger such that the syringe is rendered inoperative for further use.

The invention also provides for an injection device comprising a barrel, a plunger having a portion structured and arranged to move within the barrel, a needle support, and a locking arrangement that one of selectively locks a proximal end portion of the plunger to the needle support and automatically locks a proximal end portion of the plunger to an outer portion of the needle support.

The injection device may be at least one a syringe, a hypodermic syringe, a single-use syringe, and a retractable syringe. The barrel may comprise a cylindrically shaped internal space structured and arranged to receive therein a medicine. The barrel may further comprise a proximal opening within which the needle support is at least one of press fit and in sealing engagement therewith. The plunger may comprise a piston arranged at a proximal area of the plunger and an actuating flange arranged at a distal area of the plunger. The plunger may further comprise at least one of the piston being an elastomer or rubber piston, a frangible section adapted to allow the plunger to be broken in two, a stop flange adapted to limit forward movement of the plunger into the barrel, and a stop projection adapted to limit rearward movement of the plunger out of the barrel. The barrel, the plunger, and the needle support each comprise a synthetic resin material. The needle support may comprise a generally cylindrical distal portion and a needle projecting from a proximal end portion. The needle may comprise at least one of metal and stainless steel. The barrel may comprise at least one of a flange arranged at a distal end, a reduced diameter portion arranged at a proximal end, an internal taper arranged at a proximal end of the barrel adapted to facilitate insertion of the needle support, and at least one internal sealing projection arranged within a proximal end opening of the barrel adapted to sealingly engage an outer surface of the needle support.

The locking arrangement may selectively lock a proximal end portion of the plunger to the needle support. The locking arrangement may selectively lock the proximal end portion of the plunger to the needle support upon rotation of the plunger. The locking arrangement may selectively lock the proximal end portion of the plunger to the needle support only upon rotation of the plunger. The locking arrangement may selectively lock the proximal end portion of the plunger to the needle support upon rotation of the plunger to a predetermined position. The locking arrangement may selectively lock the proximal end portion of the plunger to the needle support upon rotation of the plunger to a position approximately transverse to an initial unlocked position. The plunger may be movable to a full insertion or to a full injection position without causing the proximal end portion of the plunger to become locked to the needle support. The plunger may be movable to an injection position which minimizes a dead space without causing the proximal end portion of the plunger to become locked to the needle support. The plunger may be movable to an injection position which causes contact between a piston of the plunger and an internal shoulder of the barrel without causing the proximal end portion of the plunger to become locked to the needle support. The plunger may be movable to an injection position which causes contact between a portion of the plunger and an internal shoulder of the barrel without causing the proximal end portion of the plunger to become locked to the needle support.

The proximal end portion of the plunger may comprise at least one of a generally flat projecting portion with oppositely arranged projections adapted to engage, in the locked position, with internal projecting shoulders of the needle support, a generally flat projecting portion with at least one projection adapted to engage, in the locked position, with at least one internal projecting shoulder of the needle support, a projecting portion comprising opposite facing planar surfaces and oppositely arranged projections adapted to engage, in the locked position, with internal projecting shoulders of the needle support, a projecting portion comprising opposite facing planar surfaces and at least one projection adapted to engage, in the locked position, with at least one internal projecting shoulder of the needle support, a projecting portion comprising at least one tapered projection adapted to engage, in the locked position, with at least one internal projecting shoulder of the needle support, a projecting portion comprising at least one fin-shaped projection adapted to engage, in the locked position, with at least one internal projecting shoulder of the needle support, and a generally cylindrical projecting portion comprising at least one fin-shaped projection adapted to engage, in the locked position, with at least one internal projecting shoulder of the needle support.

The locking arrangement may automatically lock a proximal end portion of the plunger to an outer portion of the needle support. The locking arrangement may automatically lock the proximal end portion of the plunger to the outer portion of the needle support when the plunger is moved to at least one of a full insertion position and a full injection position.

A distal end portion of the needle support may comprise at least one of a generally cylindrical opening sized to receive therein the proximal end portion of the plunger and at least one internal projecting shoulder adapted to engage, in the locked position, with at least one projection of the proximal end portion, a generally cylindrical opening sized to receive therein the proximal end portion of the plunger and two oppositely arranged internal projecting shoulders adapted to engage, in the locked position, with at least one projection of the proximal end portion, a generally cylindrical opening sized to receive therein the proximal end portion of the plunger and a generally rectangular-shaped distal entrance opening, and a generally cylindrical opening sized to receive therein the proximal end portion of the plunger and a generally double D-shaped distal entrance opening.

The invention also provides for a syringe comprising a barrel, a plunger having a portion structured and arranged to move within the barrel, a needle support, and a locking arrangement that one of locks a proximal end portion of the plunger to the needle support when the plunger is rotated from an unlocked position to a locked position and locks the proximal end portion of the plunger to an outer portion of the needle support.

The invention also provides for a syringe comprising a barrel comprising a main internal chamber for housing a medicine, a plunger having a piston structured and arranged to move within the main internal chamber and thumb engaging flange arranged outside the barrel, a needle support, and a locking arrangement that is structured and arranged to one of lock a proximal end portion of the plunger to the needle support when the plunger is rotated from a first position to a second position while being in a full injection position and lock a proximal end portion of the plunger to an outer portion of the needle support after being moved to a full injection position, wherein, when the plunger is in the full injection position, the syringe has essentially no dead space between the piston and an internal shoulder of the barrel.

The invention also provides for a method of using any of the injection devices described above, wherein the method comprises moving the plunger away from the needle support so as to cause a desired amount of medicine to enter into the barrel, moving the plunger towards the needle support so as to cause medicine to exit the barrel through the needle support, and locking the proximal end portion of the plunger to the needle support by rotating the plunger from a first position to a second position.

The method may further comprise, after the locking, moving the plunger away from the needle support so as to cause the needle support to be fully positioned within the barrel.

The invention also provides for a method of using any of the injection devices described above, wherein the method comprises moving the plunger away from the needle support so as to cause a desired amount of medicine to enter into the barrel, moving the plunger towards the needle support so as to cause medicine to exit the barrel through the needle support and so as to position the plunger at least one of at a full injection position and at a position substantially eliminating a dead space in the syringe, again moving the plunger away from the needle support, and locking the proximal end portion of the plunger to the needle support by rotating the plunger from a first position to a second position.

The method may further comprise, after the locking, moving the plunger away from the needle support so as to cause the needle support to be fully positioned within the barrel.

The invention also provides for a method of using any of the injection devices described above, wherein the method comprises moving the plunger away from the needle support so as to cause a desired amount of medicine to enter into the barrel, moving the plunger towards the needle support so as to cause medicine to exit the barrel through the needle support, and locking the proximal end portion of the plunger to an outer portion of the needle support after the plunger is moved to a full injection position.

The method may further comprise, after the locking, moving the plunger away from the needle support so as to cause the needle support to be fully positioned within the barrel.

The invention also provides for a method of using any of the injection devices described above, wherein the method comprises moving the plunger away from the needle support so as to cause a desired amount of medicine to enter into the barrel, moving the plunger towards the needle support so as to cause medicine to exit the barrel through the needle support and so as to position the plunger at least one of at a full injection position and at a position substantially eliminating a dead space in the syringe, again moving the plunger away from the needle support, and locking the proximal end portion of the plunger to an outer portion of the needle support after the plunger is moved to a full injection position.

The method may further comprise, after the locking, moving the plunger away from the needle support so as to cause the needle support to be fully positioned within the barrel.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a cross-section view of a prior art syringe. The syringe is shown in a prior-use or packaged position and is ready to receive therein a medicine;

FIG. 2 shows a cross-section view of the prior art syringe of FIG. 1. The syringe is shown moving to filling position. This would occur when the needle is inserted into a medicine bottle and the user begins to move the plunger back in order to place the desired amount of medicine into the syringe;

FIG. 3 shows a cross-section view of the prior art syringe of FIG. 2 after the syringe has assumed the used position. This would occur when the needle is inserted into tissue and the user moves move the plunger forwards in order to inject the medicine into the patient. As is evident from FIG. 3, movement of the plunger to the full injection position causes a front end portion of the plunger to become locked the needle support. The user need only move the plunger backwards to cause the needle support to retract into the syringe barrel so as to become fully housed therein;

FIG. 4 shows a cross-section view of a syringe according to one embodiment of the invention. The syringe is shown in a prior-use or packaged position and is ready to receive therein a medicine;

FIG. 5 shows a cross-section view of the syringe of FIG. 4. The syringe is shown after the plunger has moved to a desired filling position. This would occur when the needle is inserted into a medicine bottle and the user begins to move the plunger back in order to place the desired amount of medicine into the syringe;

FIG. 6 shows a cross-section view of the syringe of FIG. 4 after the syringe has assumed the used position. This would occur when the needle is inserted into tissue and the user moves the plunger forwards in order to inject the medicine into the patient. As is evident from FIG. 6, movement of the plunger to the full injection position causes a front end portion of the plunger to be inserted into the needle support by substantially the same amount as was the case in FIG. 4. No locking between the plunger and the needle support has occurred. In the position shown in FIG. 6, the user can move the plunger backwards without causing the needle support to retract into the syringe barrel;

FIG. 7 shows a cross-section view of the syringe of FIG. 6 after the syringe has assumed the locked position. This would occur when the user rotates the plunger (i.e., by about 90 degrees) from the position shown in FIG. 6 to the position shown in FIG. 7. As is evident from FIG. 7, rotation of the plunger causes the projection shoulders of the front end portion of the plunger to be aligned with inwardly projecting shoulders of the needle support. The plunger and the needle support are now locked together. In the position shown in FIG. 7, the user can move the plunger backwards thereby causing the needle support to retract into the syringe barrel;

FIG. 8 shows a cross-section view of the syringe of FIG. 7 from another perspective that is 90 degrees to the perspective shown in FIG. 7. As is evident from FIG. 8, the projection shoulders of the front end portion of the plunger are aligned with inwardly projecting shoulders of the needle support. The plunger and the needle support are locked together;

FIG. 9 shows a cross-section view of the syringe of FIG. 8 after the plunger has been moved to a fully retracted position thereby placing the needle support within the syringe barrel;

FIG. 10 shows a cross-section view of the syringe of FIG. 9 after the plunger has been deflected to the point where it breaks leaving the portion with the piston and locked to the needle support within the syringe barrel;

FIG. 11 shows a cross-section view of the syringe barrel used in the syringe shown in FIG. 4;

FIG. 12 shows a rear view of the syringe barrel shown in FIG. 11;

FIG. 13 shows a front view of the syringe barrel shown in FIG. 11;

FIG. 14 shows an enlarged cross-section view of the needle support used in the syringe of FIG. 4;

FIG. 15 shows a partial cross-section view of the needle support shown in FIG. 14;

FIG. 16 shows a rear view of the needle support shown in FIG. 15;

FIG. 17 shows a cross-section view of the syringe barrel and the needle support used in the syringe shown in FIG. 4. The needle support is shown in a position prior to being inserted into the front end opening of the syringe barrel;

FIG. 18 shows a side view of the plunger used in the syringe shown in FIG. 4;

FIG. 19 shows a top view (a view transverse to the view shown in FIG. 18) of the plunger shown in FIG. 18;

FIG. 20 shows a side view of the plunger and the needle support used in the syringe shown in FIG. 4. The plunger and needle support are shown in a locked position;

FIG. 21 shows another side view of the plunger and the needle support used in the syringe shown in FIG. 4. The plunger and needle support are shown in a unlocked position;

FIG. 22 shows an enlarged view of the plunger and the needle support shown in n FIG. 21, except that the plunger is rotated 90 degrees so as to show a transverse view of the unlocked position;

FIG. 23 shows another view of FIG. 22, except that the plunger is positioned in the full injection position so as to eliminate the syringe barrel dead space;

FIG. 24 shows a cross-section view of a syringe according to another embodiment of the invention. The syringe is shown in a locked position. The syringe can assume this position after injection and after the plunger is rotated to the locked position;

FIG. 25 shows a rear view of the syringe shown in FIG. 24 (from the perspective of the plunger thumb actuating flange being removed);

FIG. 26 shows a cross-section view of a syringe according to another embodiment of the invention. The syringe is shown in an unlocked position. The syringe can have this position both before use, after injection, and before the plunger is rotated to the locked position;

FIG. 27 shows a rear view of the syringe shown in FIG. 26 (from the perspective of the plunger thumb actuating flange being removed);

FIG. 28 shows an enlarged cross-section view of a syringe according to another embodiment of the invention. The syringe is shown in an unlocked position. The syringe can assume this position prior to use;

FIG. 29 shows another enlarged cross-section view of FIG. 28 and illustrates an intermediate position as the plunger moves towards the locked position. The syringe can assume this position during or after injection;

FIG. 30 shows another enlarged cross-section view of FIG. 28 and illustrates a locked position of the plunger and needle support. The syringe can assume this position after injection;

FIG. 31 shows another enlarged cross-section view of FIG. 28 and illustrates how the plunger can cause the needle support (which is locked thereto) to be retracted into the syringe barrel;

FIG. 32 shows an enlarged cross-section view of a syringe according to still another embodiment of the invention. The syringe is shown in an unlocked position. The syringe can assume this position prior to use;

FIG. 33 shows another enlarged cross-section view of FIG. 32 and illustrates a locked position of the plunger and needle support. The syringe can assume this position after injection;

FIG. 34 shows an enlarged cross-section view of a syringe according to still another embodiment of the invention. The syringe is shown in an unlocked position. The syringe can assume this position prior to use;

FIG. 35 shows a rear view of the needle support which can be used in the syringe shown in FIG. 34;

FIG. 36 shows a rear view of the needle support shown in FIG. 35 and illustrates a cross-section of the front end of the plunger. This figure illustrates how the front end of the plunger is inserted in the needle support and shows a rear view of the unlocked position;

FIG. 37 shows an enlarged cross-section view of a syringe according to still another embodiment of the invention. The syringe is shown in an unlocked position. The syringe can assume this position prior to use;

FIG. 38 shows a rear view of the needle support which can be used in the syringe shown in FIG. 37; and

FIG. 39 shows a rear view of the needle support shown in FIG. 38 and illustrates a cross-section of the front end of the plunger. This figure illustrates how the front end of the plunger is inserted in the needle support and shows a rear view of the unlocked position.

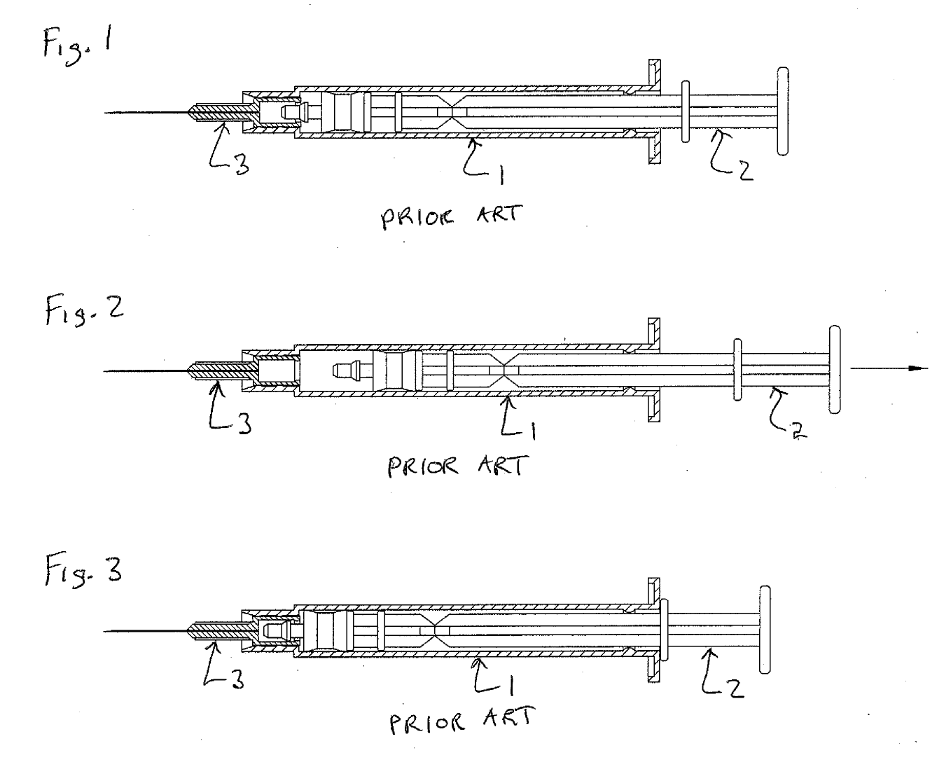

### DETAILED DESCRIPTION OF THE INVENTION

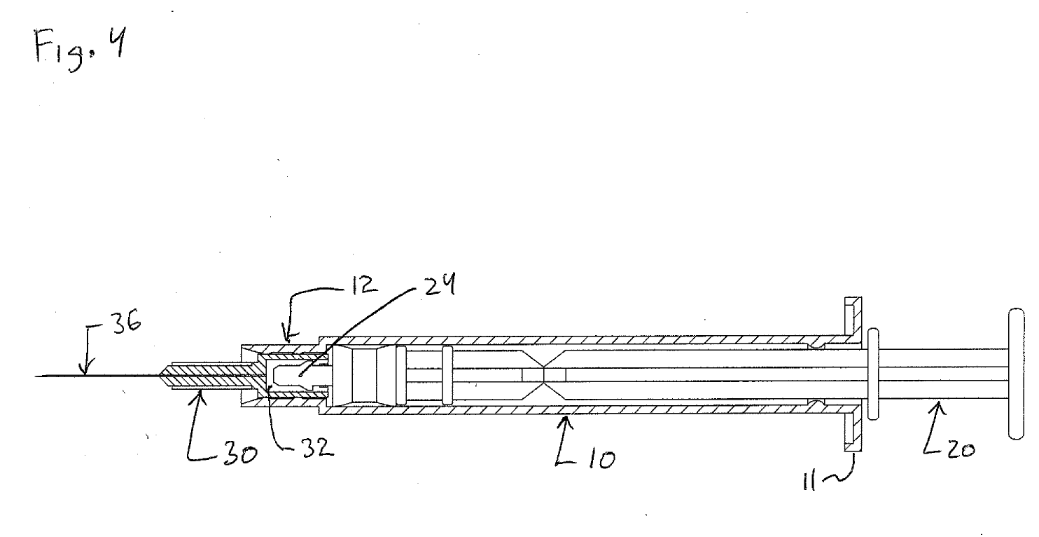

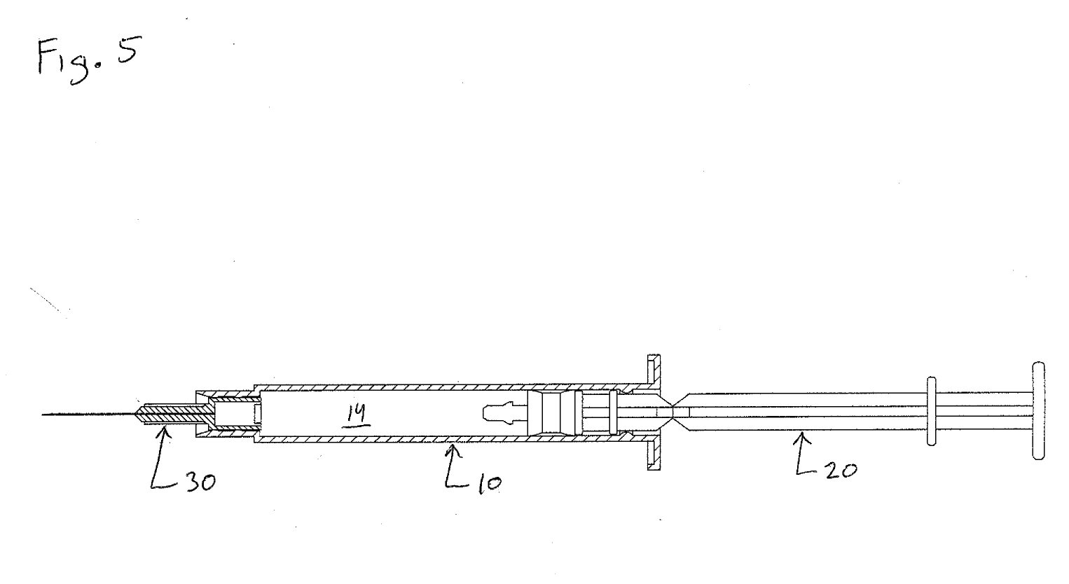

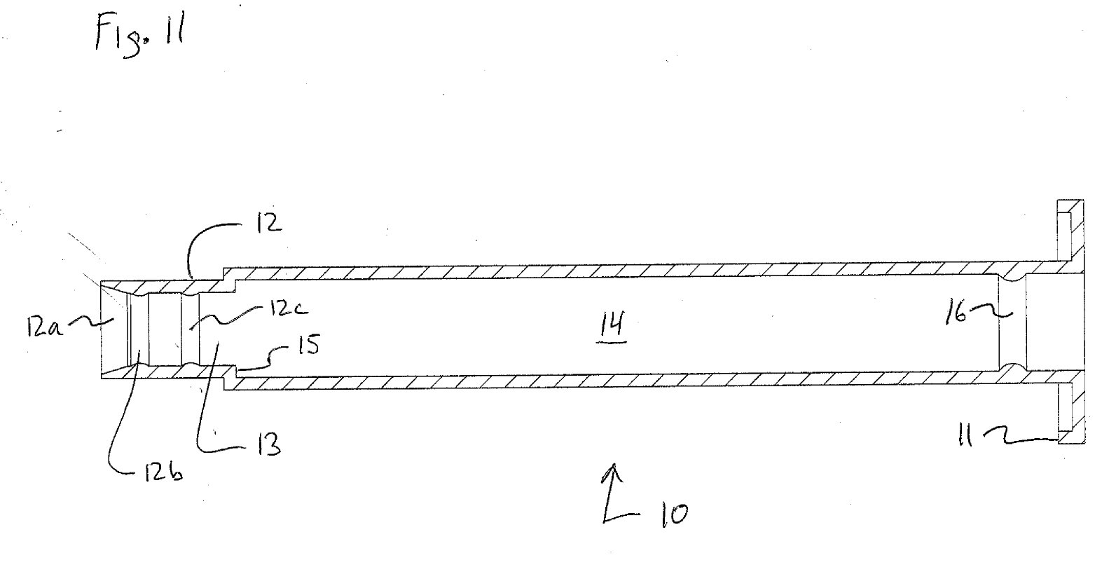

Referring now to the drawings and first to FIG. 4 which shows a first embodiment of a retractable hypodermic syringe. The syringe includes a generally elongate cylindrical barrel 10 having a transverse flange 11 arranged at a distal end of the barrel 10. The proximal end of the barrel 10 includes a reduced diameter section 12 which forms a tubular extension and/or a cylindrical sleeve which is disposed concentrically about a proximal end passage 13 (see FIG. 11) that extends through the section 12 and into a main internal chamber 14 defined by the main cylindrical section of the barrel 10.

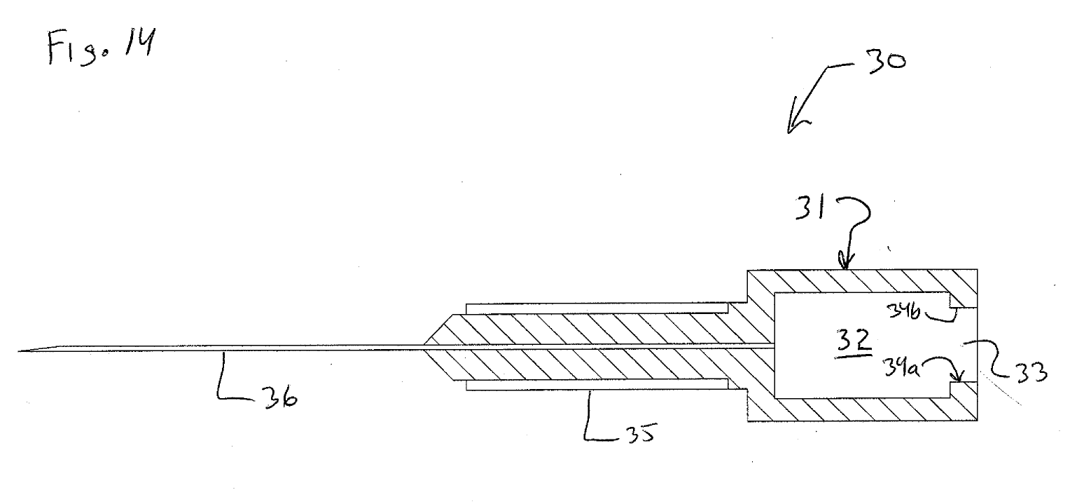

A needle support 30 is normally positioned within the passage 13 (see FIG. 11) and includes a rear end portion 31 (see FIG. 14) which is sealed to and/or in frictional engagement with the cylindrical section 12. As shown in FIG. 1, the rear end portion 31 of the needle support 30 includes a main opening 32 configured to receive therein a front or proximal end portion 24 of the plunger 20.

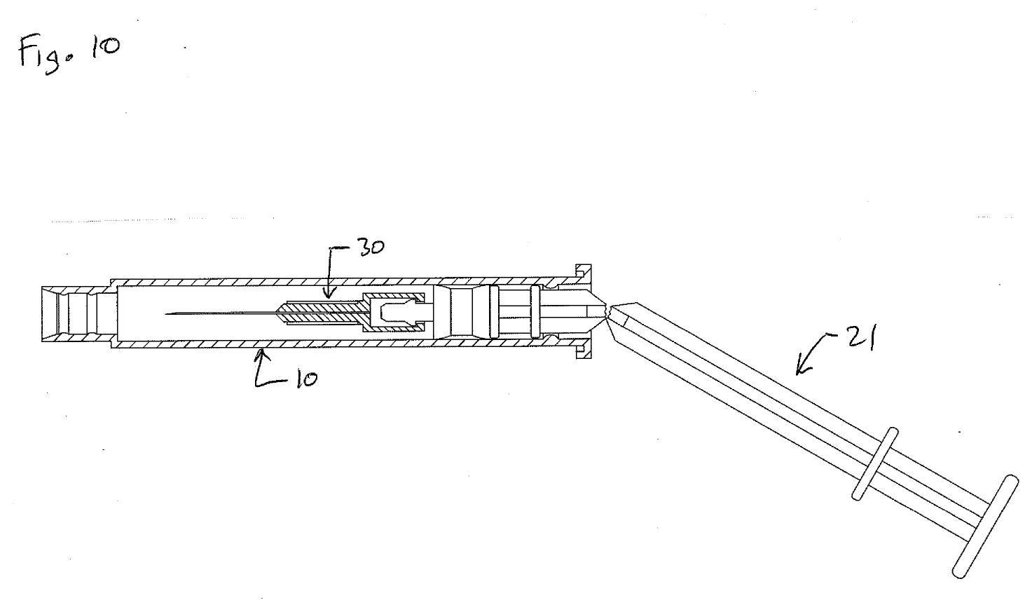

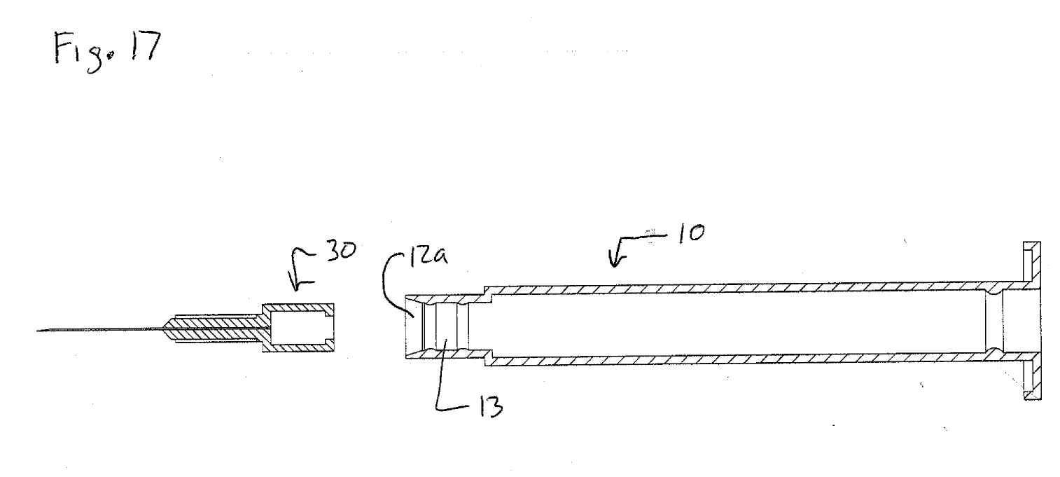

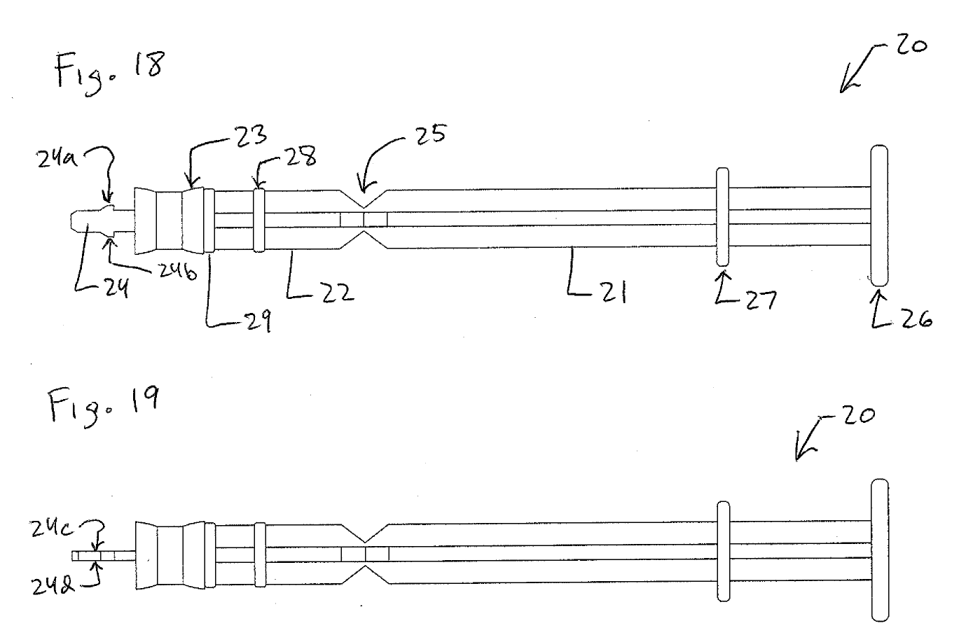

Arranged within the barrel 10 of the syringe is movably disposed a plunger 20. The plunger 20 can, by way of non-limiting example, be formed by intersecting webs of polymer material which may be molded in integral assembly to provide a light-weight plunger having sufficient structural integrity for loading the barrel 10 with medicament and dispensing the medicament through the passage 14 and hollow needle 36 into the body tissues of the patient. The plunger 20 is also of sufficient structural integrity for imparting rotational movement to the needle support 30 as will be described below for the purpose of locking and unlocking the plunger to the needle support 30 relative to the barrel 10. The plunger 20 is provided with an end flange 26 which is typically engaged by the thumb of the user while the transverse flange 11 is engaged by the fingers of the user in order to enable the plunger 20 to be forced into the barrel 10 for the purpose of expelling the medicament from the barrel 10 through the passage 14. The plunger 20 also utilizes weakened portions of the webs to form a frangible section 25 (see FIGS. 18 and 19) which enables the distal section 21 of the plunger 20 to be broken away from the proximal section 22 in the manner shown in FIG. 10. The frangible section 25 may also be scored to insure that it will break at the proper location. The section 25, of course, should be of appropriate dimension in order to provide sufficient structural integrity to the plunger 20 to enable the plunger 20 to transmit locking and unlocking forces to the needle support 30 and to impart linear force to the plunger 20.

FIG. 4 shows the syringe in a prior-use or packaged position and is ready to receive therein a medicine and illustrates how the portion 24 is inserted within the opening 32 of the needle support 30 without causing the plunger 20 to become locked to the needle support 30.

FIG. 5 shows the syringe of FIG. 4 after the plunger 20 has moved (i.e., been retracted) to a desired filling position. This would occur when, e.g., the needle 36 is inserted into a medicine bottle and the user begins to move the plunger 20 rearward or back in order to place the desired amount of medicine into the syringe.

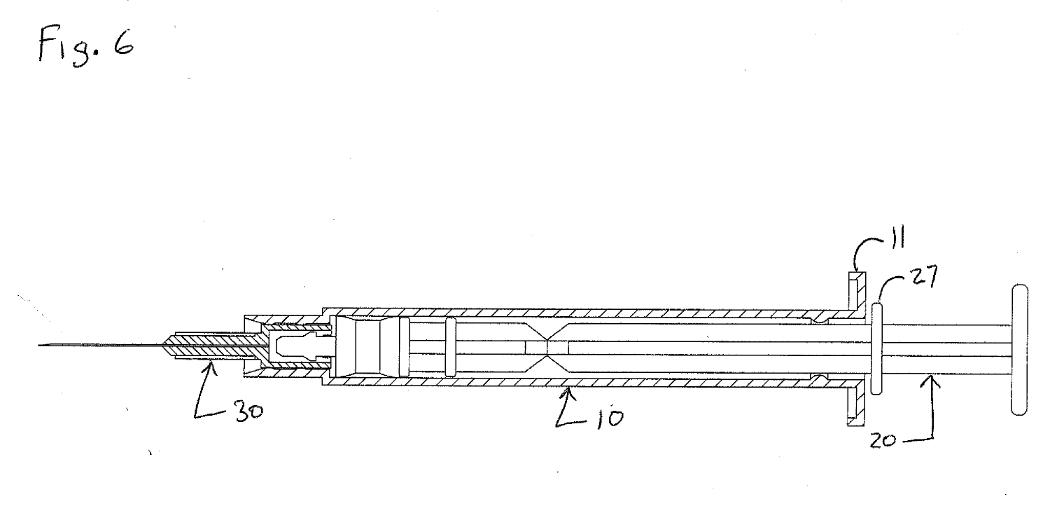

FIG. 6 shows the syringe of FIG. 4 after the syringe has assumed the used or injection position. This would occur when the needle 36 is inserted into tissue and the user moves the plunger 20 forwards in order to inject the medicine into the patient. As is evident from FIG. 6, movement of the plunger 20 to the substantially full injection position causes the front end or proximal end portion 24 of the plunger 20 to be inserted into the needle support 30 by substantially the same amount as was the case in FIG. 4. Furthermore, no automatic locking has occurred between the plunger 20 and the needle support 30. In the position shown in FIG. 6, the user can move the plunger 20 backwards without causing the needle support 30 to become unlocked to and/or un-engaged with the section 12 and retract into the syringe barrel 10. The full injection position of the syringe can be defined by contact between plunger flange 27 and the barrel flange 11 and/or by contact between the leading edge of the plunger piston 23 and the bottom shoulder 15 of the barrel 10. In the latter case, the dead-space of the syringe is effectively and/or substantially eliminated or minimized.

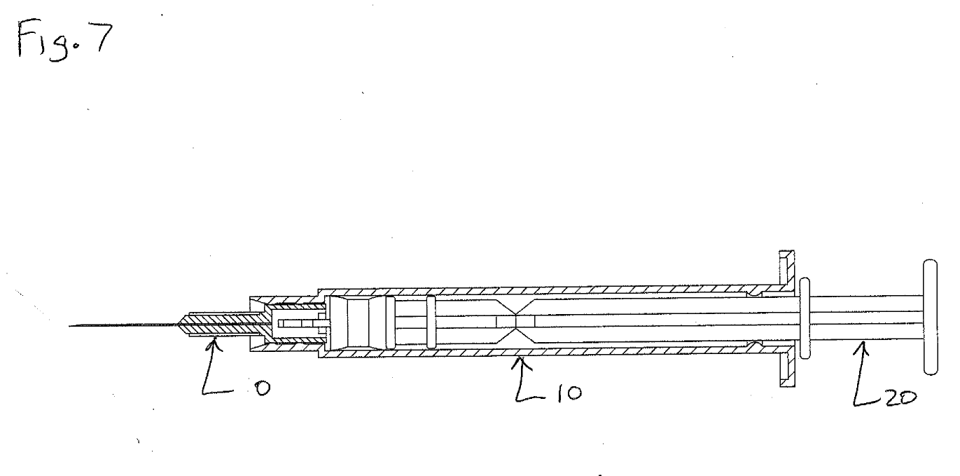

FIG. 7 shows the syringe of FIG. 6 after the syringe has assumed the locked position. This would occur when the user rotates the distal flange 26 of the plunger 20 (e.g., by about 90 degrees) from the position shown in FIG. 6 to the position shown in FIG. 7. Of course, the invention is not limited to rotational movements which are 90 degrees and can be any desired rotational angle. To ensure that the user can easily perform this rotation movement relative to the barrel 10, the outer circumferential surface of the flange 26 can be provided with a high friction surface (not shown) which take the form of e.g., a knurl, a textured surface, a high friction coating made of e.g., rubber or silicone. The invention also contemplates making some portion of all of the outer cylindrical circumferential surface of the barrel 10 or the flange 11 with a similar high friction surface (not shown). As is evident from FIG. 7, rotation of the plunger 20 causes the projection shoulders 24a and 24b (see FIG. 18) of the front end portion 24 of the plunger 20 to become aligned with inwardly projecting shoulders 34a and 34b (see FIG. 14) of the needle support 30. The plunger 20 and the needle support 30 are now locked together. That is, in this position, movement of the plunger 20 rearward will cause engagement between the projection shoulders 24a and 24b and the inwardly projecting shoulders 34a and 34b thereby ensuring that axial movement of the plunger 20 will translate to corresponding axial movement of the needle support 30. In the position shown in FIG. 7, the user can, ant any desired time, move the plunger 20 backwards thereby causing the needle support 30 to retract into the syringe barrel 10.

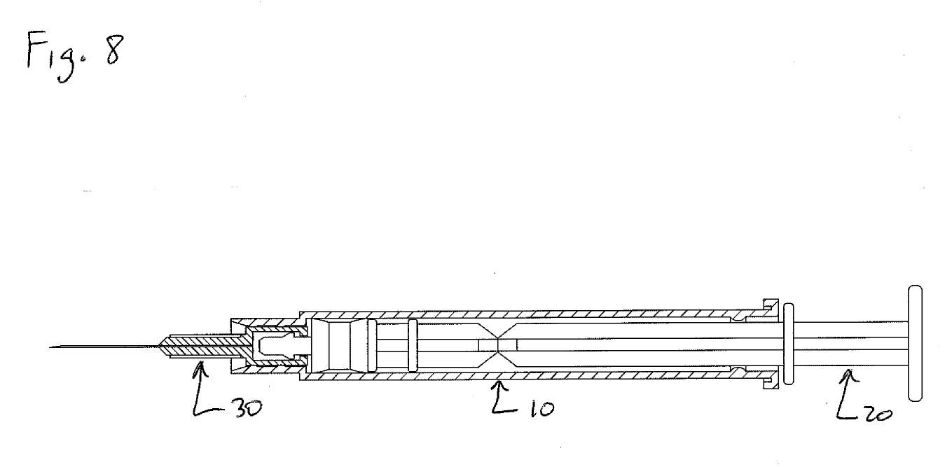

FIG. 8 shows the syringe of FIG. 7 from a perspective that is 90 degrees to the perspective shown in FIG. 7. As is evident from FIG. 8, the projection shoulders 24a and 24b of the front end portion 24 of the plunger 20 are aligned with inwardly projecting shoulders 34a and 34b of the needle support 30. The plunger 20 and the needle support 30 are, as explained above, locked together.

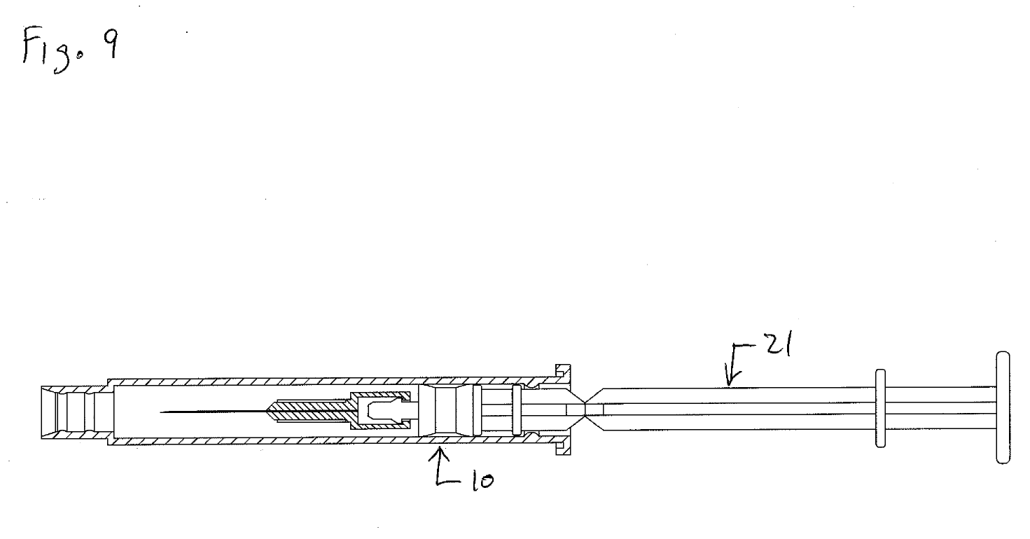

FIG. 9 shows the syringe of FIG. 8 after the plunger 20 has been moved to a fully retracted position, i.e., defined by engagement with or contact between circular projection or flange 28 (see FIG. 18) and internal circular projection 16 (see FIG. 11), thereby positioning the needle support 30 fully within the syringe barrel 10. In this position, it is nearly impossible for anyone coming in contact with the syringe to get pricked by the needle 36.

FIG. 10 shows the syringe of FIG. 9 after the plunger 10 has been deflected to the point where it breaks leaving the proximal portion 22 with the piston locked to the needle support 30 within the syringe barrel 10. The breaking of the plunger 20 occurs, of course, at the frangible section 25. As is evident from FIG. 10, once the distal section 21 is removed, the syringe is rendered entirely unusable and can be safely disposed of and/or handled without concern that anyone handling the syringe will be pricked by the needle 36 by accident.

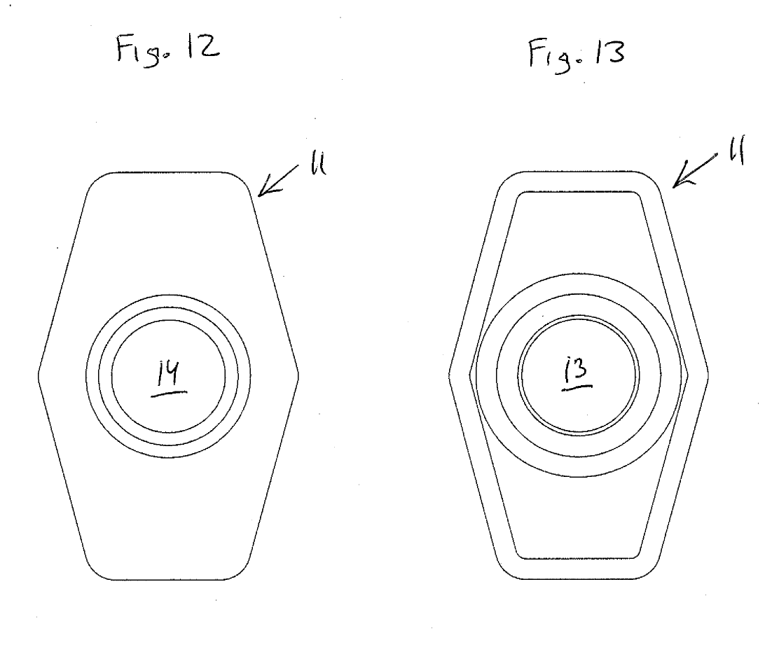

FIG. 11 shows the syringe barrel 10 used in the syringe shown in FIG. 4. The syringe barrel 10 is a generally elongate cylindrical barrel having a flange 11 arranged at a distal end of the barrel 10. As is shown in FIGS. 12 and 13, the flange 11 can a polygonal shape. Of course, the invention also contemplates other shapes such as, e.g., circular, oval, as well as other polygonal shapes. The proximal end of the barrel 10 includes a reduced diameter section 12 which forms a tubular extension and/or a cylindrical sleeve. The section 12 is disposed concentrically about a generally cylindrical proximal end passage 13 that extends through the section 12 into a main internal chamber 14 defined by the main cylindrical section of the barrel 10. The proximal end of the barrel 10 includes an internal tapered section 12a which facilitates insertion of the needle support 30 from the proximal end. In order to prevent any medicine from leaking out of the syringe and to ensure that the needle support 30 is axially constrained so that the syringe can be used without the needle support 30 moving prematurely, the section 12 includes at least one and preferably two or more circular spaced-apart projections 12b and 12c which are configured to frictionally and/or sealingly engage with the outer cylindrical surface of the needle support 30. The invention, however, contemplates other mechanisms for ensuring that the syringe can be used without the needle support 30 moving prematurely. For example, it is contemplated that the needle holder 30 can instead be provided with one or more circular projections which frictionally engage a generally cylindrical inner surface of the section 12. The barrel 10 also includes a bottom shoulder 15 which when contacted by the leading end of the piston 23 ensures that the syringe has substantially no dead space. This can be particularly important when the medicine is very expensive and/or rare and one seeks to minimize any waste thereof. This can also be particularly important when the medicine must be administered in very precise dosages. The barrel 10 also includes a circular projection 16 which serves to prevent the plunger 20 from being move rearwardly beyond a desired point. The projection 16 also serves to locate the frangible section 25 is a predetermined position outside of the barrel 10 to ensure that the distal section 21 will break with the least amount of effort.

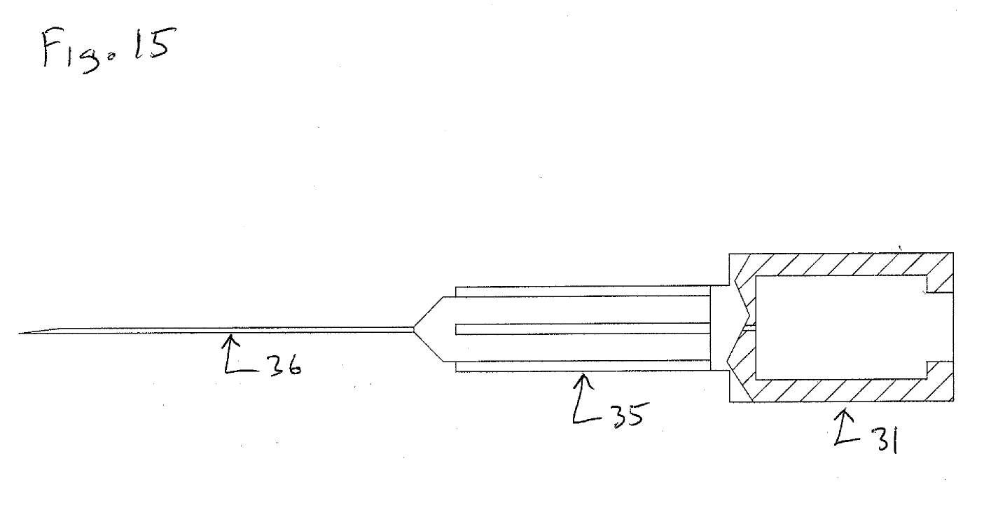

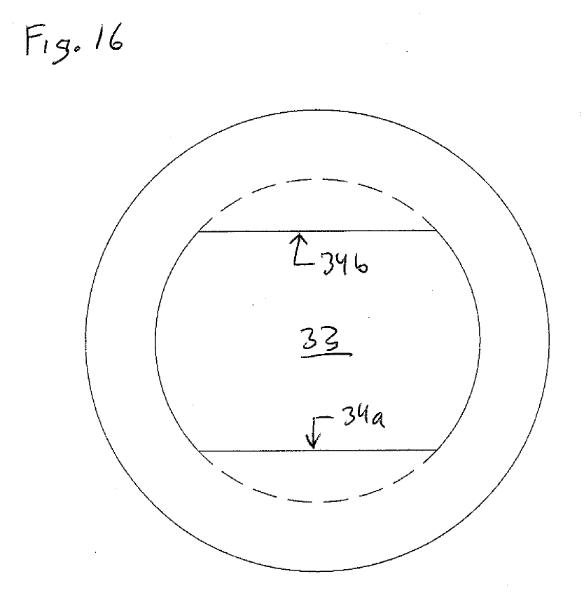

FIGS. 14-16 show the needle support 30 which is used in the embodiment shown in FIG. 4. The needle support 30 includes a distal section 31 which is sized and configured to be press-fit inside the passage 13. As was explained above, the rear end portion 31 is preferably sealed to and/or in frictional engagement with the cylindrical section 12 so as to ensure that the syringe does not leak and so that the needle support 30 is prevented from axial movement until being so moved by the plunger 20. The rear end portion 31 of the needle support 30 includes a main opening 32 configured to receive therein a front or proximal end portion 24 of the plunger 20. However, in order for the portion 24 to enter into the opening 32, the needle holder 30 also includes a key or entrance opening 33 which is defined by one or more internally projecting shoulders 34a and 34b. This opening 33 prevents the portion 24 from locking with the needle holder 30 unless the portion is oriented or rotated to a correct position. The needle support 30 also includes a proximal hollow needle 36 which extends from a smaller diameter section 35 arranged between the needle 36 and the section 31. As is apparent from FIG. 14, the needle 36 extends all the way through section 35 and provides a fluid passage communicating with opening 32. Section 35 can have any desired configuration or size and serves mainly as the portion of the needle support 30 or syringe to which a needle protection cap (not shown) is mounted. As can be seen in FIG. 16, the entrance opening 33 can have the form of a “double D” shaped opening.

FIG. 17 shows the syringe barrel 10 and the needle support 30 used in the syringe shown in FIG. 4. The needle support 30 is shown in a position prior to being inserted into the front end opening 13 of the syringe barrel 10. The needle support 30 is preferably press-fit into the opening 13 and this insertion is facilitated by the tapered section 12a.

FIGS. 18-19 show the plunger 20 that is used in the syringe shown in FIG. 4. As explained above, the plunger 20 can be formed by intersecting webs of polymer material which may be molded in integral assembly to provide a light-weight plunger having sufficient structural integrity for loading the barrel with medicament and dispensing the medicament through the passage 14 and the hollow needle 36 into the body tissues of the patient. The plunger 20 is also of sufficient structural integrity for imparting rotational movement to the needle support 30 as was explained above for the purpose of locking and unlocking the needle support 30 and cause its movement relative to the barrel 10. The plunger 20 is provided with an end flange 26 which is typically engaged by the thumb of the user in order to enable the plunger 20 to be forced into the barrel 10 for the purpose of expelling the medicament from the barrel 10 through the passage 13. Intermediate the extremities of the plunger 20 is a frangible section 25 that allows the distal end 21 of the plunger 20 to be broken off in the manner shown in FIG. 10. The frangible section 25 may also be scored to insure that it will break at the proper location. Of course, the section 25 should also be of appropriate dimension to provide sufficient structural integrity to the plunger 20 and enable the locking and unlocking forces to be imparted to the needle support 30 and also to impart linear force to the plunger piston 23.

At a forward or proximal end of the plunger 20 there is provided a transverse support or abutment flange 29 which is of a dimension less than that of the internal cylindrical surface of the barrel 10. The flange 29 provides abutting support for a resilient piston 23 which is mounted to piston support portion that is integral with the plunger 20 and extends forwardly of the piston abutment flange 29. The piston support portion (not shown) can also include an intermediate enlargement which engages with an internal recess (not shown) of the piston 23 thus serving to secure the piston 23 to the plunger 20 thereby permitting relative rotation of the piston 23 and plunger 20. The piston 23 also defines a pair of spaced opposite facing external sealing lands that establish sealing engagement with the internal cylindrical internal wall of the syringe barrel 10. Thus, as the plunger 20 moves linearly and axially within the barrel 10 the piston lands maintain sealing engagement with the inner cylindrical surface and thereby provide the plunger 20 with the capability of imparting significant hydraulic pressure to the liquid medicament which may be contained within the syringe barrel 10. Of course, the invention contemplates other plunger and piston configurations whether conventional or otherwise.

Since, in the embodiment shown in FIG. 4, it is desirable to provide a releasable connection between the plunger 20 and the needle support 30, the plunger 20 includes a proximal end portion 24 which functions as a key and allows the plunger 20 to be selectively locked to the needle support 30. In this regard, the proximal end portion 24 has a tapered leading end, one or more tapered projections 24a and 24b which can selectively engage with one or more projections 34a and 34b of the needle support 30 depending on the rotational position of the plunger 20. In the non-limiting embodiment shown in FIGS. 18 and 19, the portion 24 is a generally flat member having opposite facing generally planar sides 24c and 24d. The thickness between the sides 24c and 24d can any desired value provide that the portion 24 maintains sufficient strength to function as intended. Preferably the thickness is between approximately 25% and 75% of the overall width of portion 24 as measured in FIG. 18.

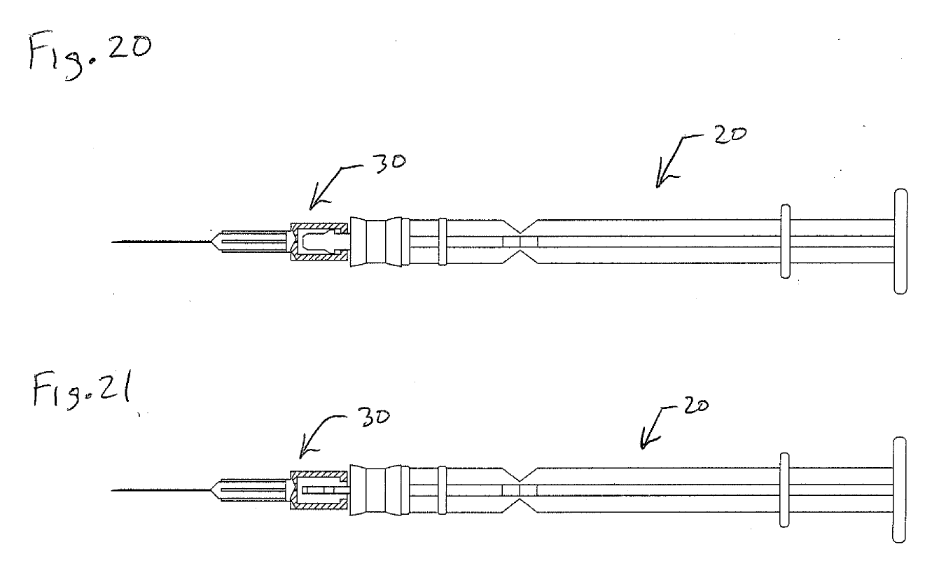

FIG. 20 shows the plunger 20 and the needle support 30 used in the syringe shown in FIG. 4. The plunger 20 and the needle support 30 are shown in a locked position. This can occur when the plunger 20 is rotated approximately 90 degrees from the position in FIG. 21.

FIG. 21 shows the plunger 20 and the needle support 30 used in the syringe shown in FIG. 4. The plunger 20 and the needle support 30 are shown in a unlocked position. This can occur when the plunger 20 is rotated approximately 90 degrees from the position in FIG. 20.

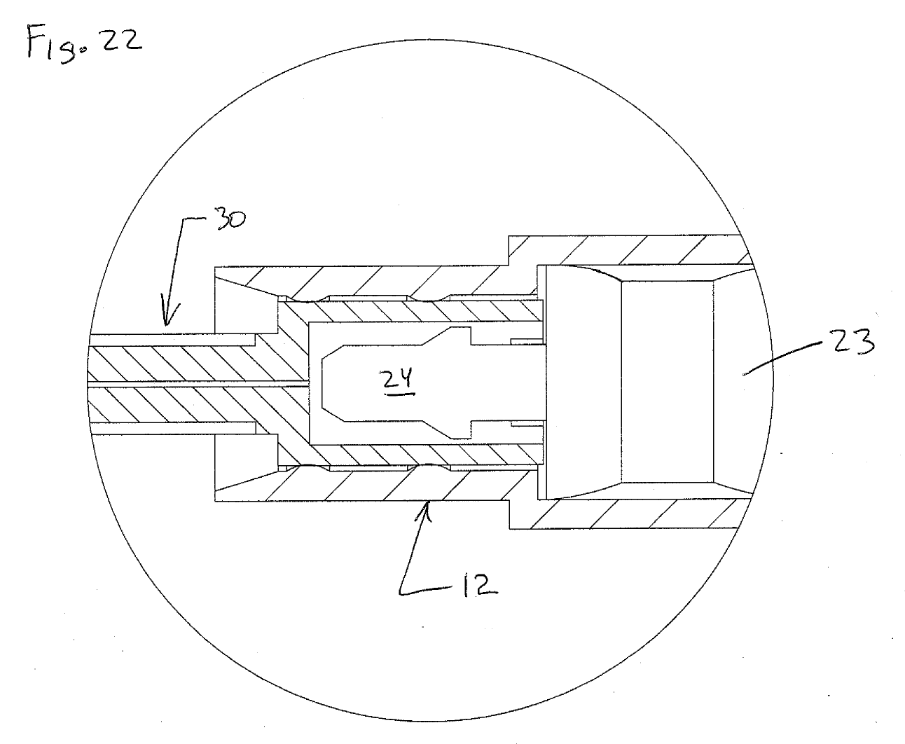

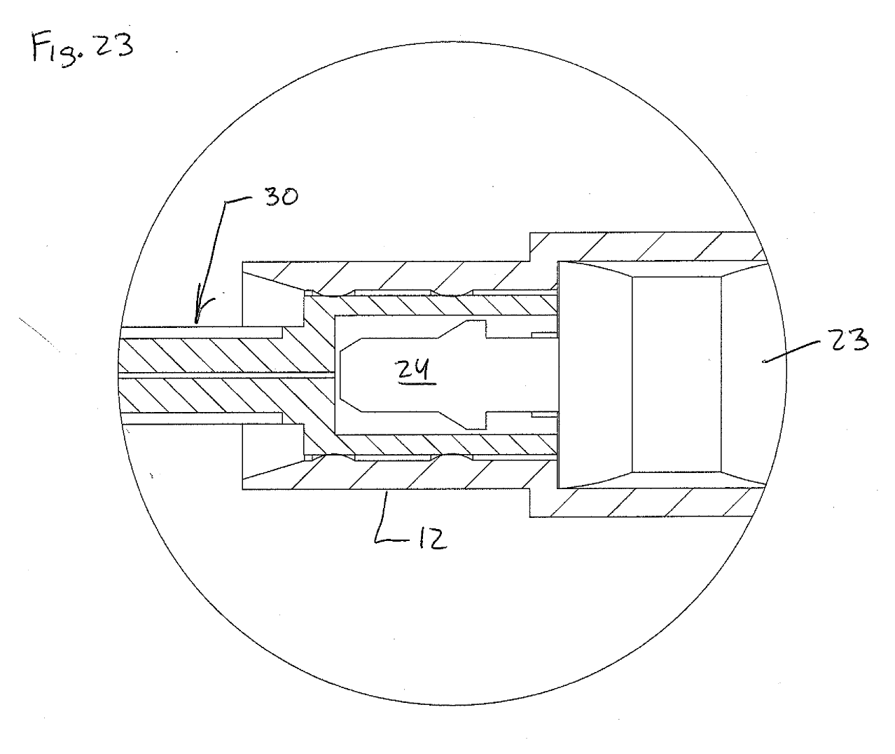

FIG. 22 shows an enlarged view of the plunger 20 and the needle support 30 shown in n FIG. 21, except that the plunger 20 is rotated 90 degrees so as to show a transverse view of the unlocked position. FIG. 23 shows another view of FIG. 22, except that the plunger 20 is positioned in the full injection position so as to eliminate the syringe barrel dead space, i.e., the leading end of the piston 23 can come into contact with shoulder 15.

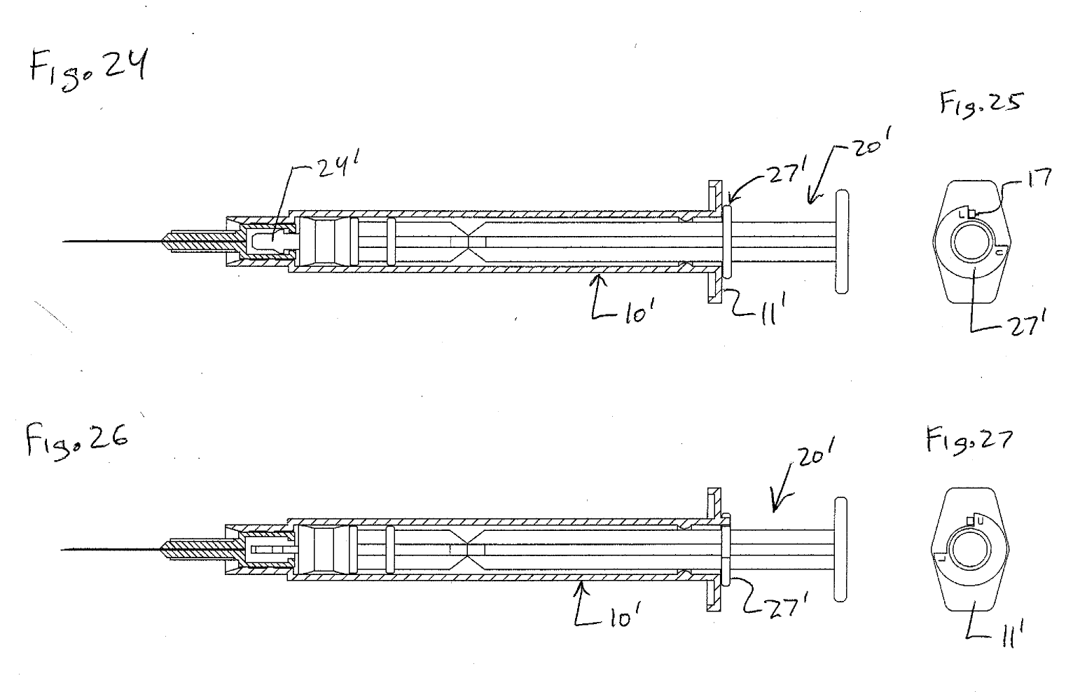

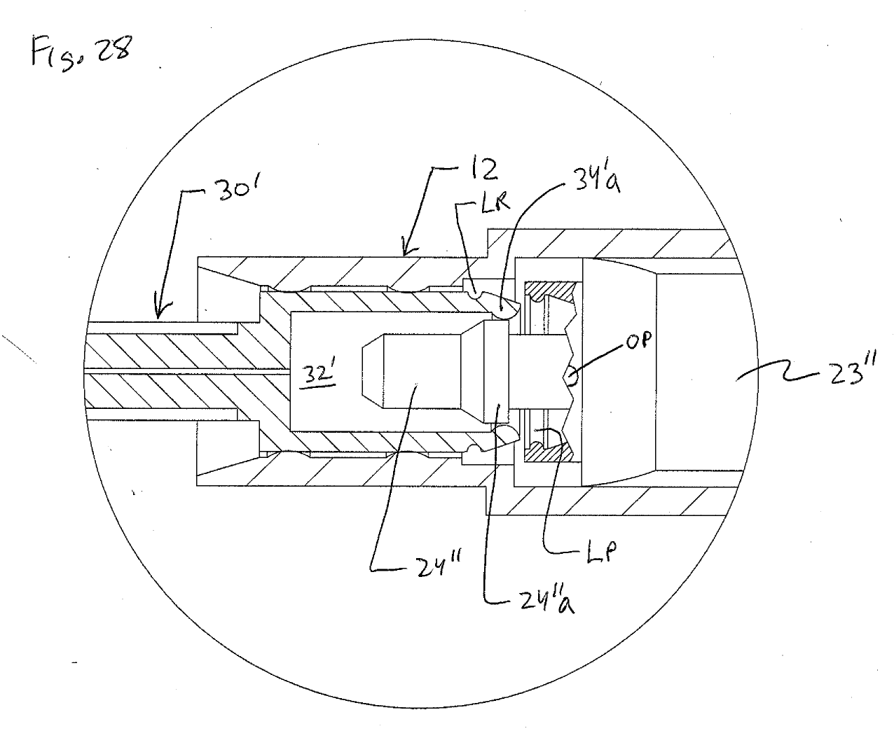

FIGS. 24-27 shows a syringe according to another embodiment of the invention. The syringe is similar to the embodiment shown in FIG. 4, except that the syringe utilizes a system for setting the plunger 20′ in each of the locked and unlocked positions. As is shown in FIGS. 25 and 27, the system utilizes a cut-away section of the flange 27′ resulting in the flange 27′ having two stop surfaces. When the plunger 20′ is rotated to the position shown in FIG. 25, one stop surface of the flange 27′ contacts a stop projection 17 integrally formed on the flange 11′ of the barrel 10′. When the plunger 20′ is rotated to the position shown in FIG. 27, another stop surface of the flange 27′ contacts the stop projection 17 integrally formed on the flange 11′ of the barrel 10′. In order to provide the user with a visual indication of the locked and unlocked positions, the flange 27′ can include symbols. For example, the symbol “L” can be used to designate when the plunger 20′ is locked to the needle support 30 and the symbol “U” can be used to designate when the plunger 20′ is unlocked to the needle support 30.

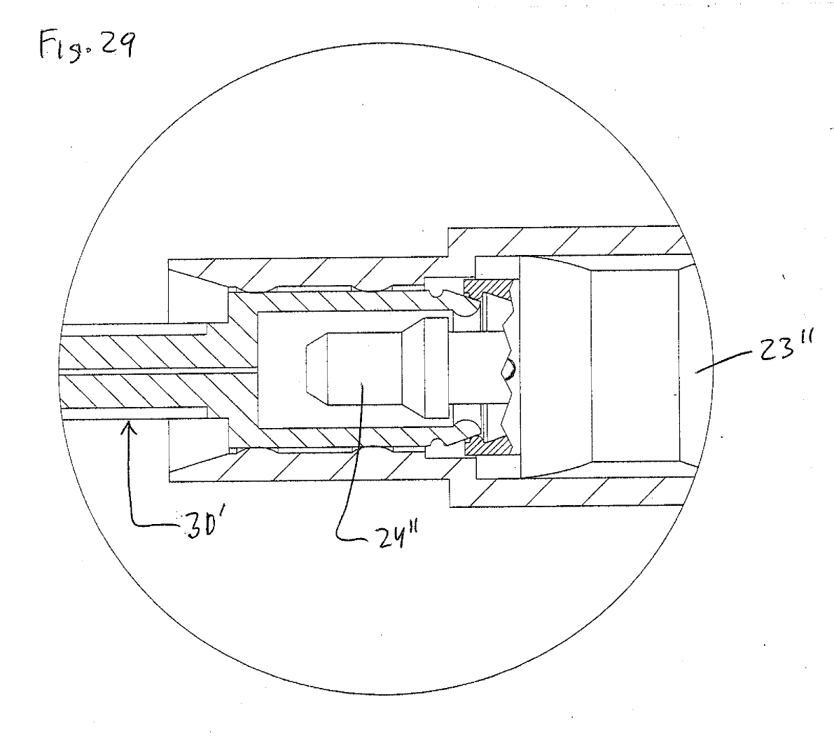

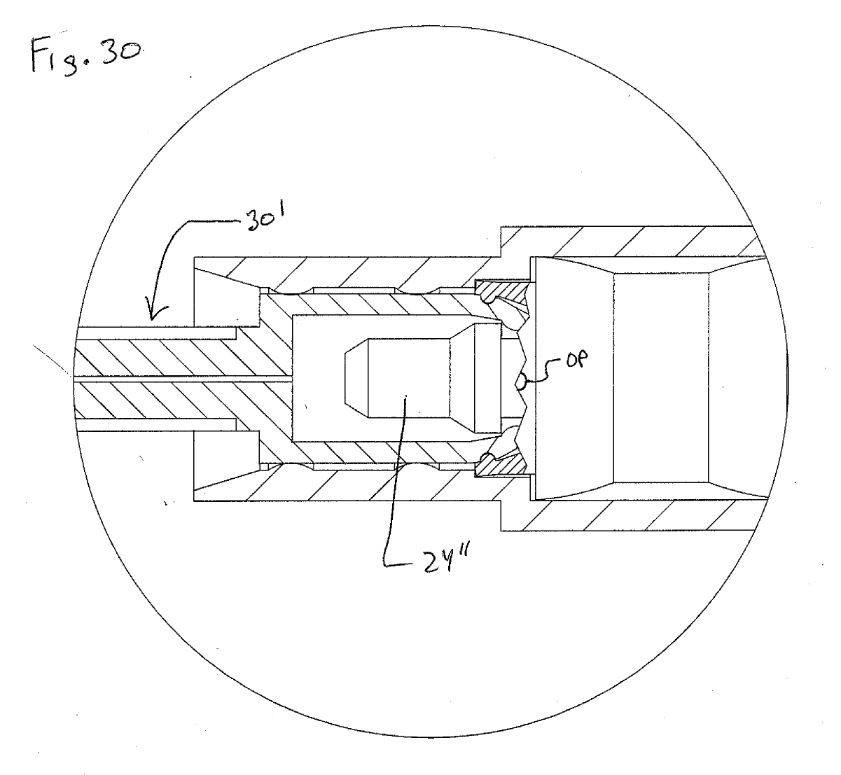

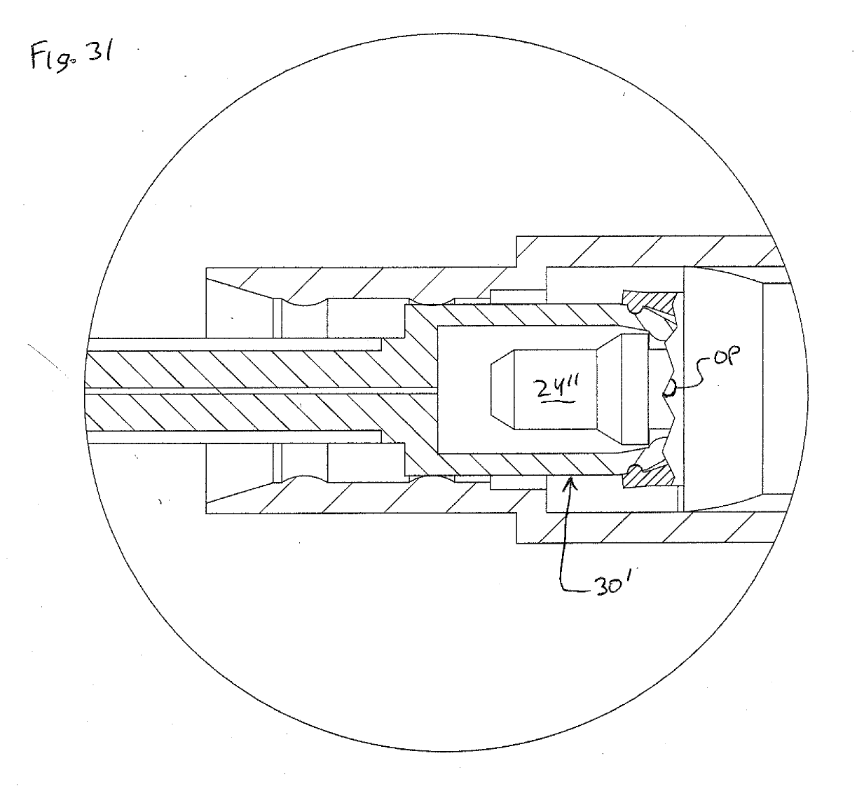

FIGS. 28-31 show a syringe according to another embodiment of the invention. In FIG. 28, the syringe is shown in an unlocked position. The syringe can assume this position prior to use. In this embodiment, the proximal end portion 24″ of the plunger is a generally cylindrically shaped member with a tapered free end to allow for easy insertion into the opening 32′ of the needle support 30′. Furthermore, the portion 24″ utilizes a circumferential tapered projection 24″a which, when the plunger is in the locked position (i.e., the full injection position), is configured to engage with a circumferential inwardly projecting rib 34′a. However, as long as the plunger does not assume the substantially fully injection position (see FIG. 30), the portion 24″ will not become locked to the needle support 30′. In order to lock the plunger to the needle support 30′, the embodiment shown in FIGS. 28-31 utilizes a sleeve-shaped plunger locking member which includes an inwardly facing circular locking projection LP is configured to lock with a circular locking recess LR arranged on an exterior surface of the needle support 30′. In order to ensure that the medicine is not prevented from exiting the syringe when the plunger is nearing the full injection position, the sleeve-shaped plunger locking member includes a plurality of through openings OP. These openings OP can be equally spaced and can be any number between, e.g., 1 and 12. Alternatively or in addition to the openings OP, the sleeve-shaped plunger locking member can include slots so as to form a plurality of spaced apart fingers each having a portion of the locking projection LP. As is evident from FIGS. 28-31, the plunger becomes locked to the needle support 30′ when the locking projection LP is received in the locking recess LR. FIG. 29 illustrates an intermediate position as the plunger moves towards the locked position. The syringe can assume this position during or after injection. FIG. 30 illustrates a locked position of the plunger and needle support 30′. As is evident from FIG. 30, the distal end of the needle support 30′ is forced to deflect inwardly as the sleeve-shaped plunger locking member moves over the distal end. Although not shown, the distal end of the needle support 30′ in the area of the locking recess LR can include slots so as to form a plurality of spaced apart fingers each having a portion of the locking recess LR. This would more easily allow the distal end to deflect inwardly and facilitate locking. FIG. 30 illustrates that when the plunger is locked to the needle support 30′, the syringe has substantially no dead space. FIG. 31 illustrates how the plunger can cause the needle support 30′ (which is locked in a permanent or non-releasable manner thereto) to be retracted into the syringe barrel.

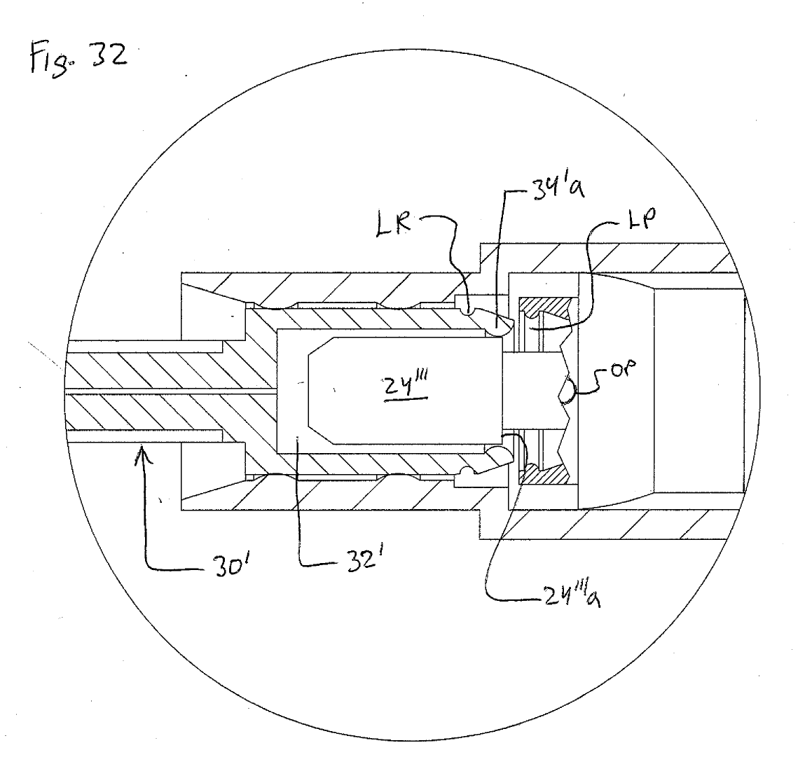

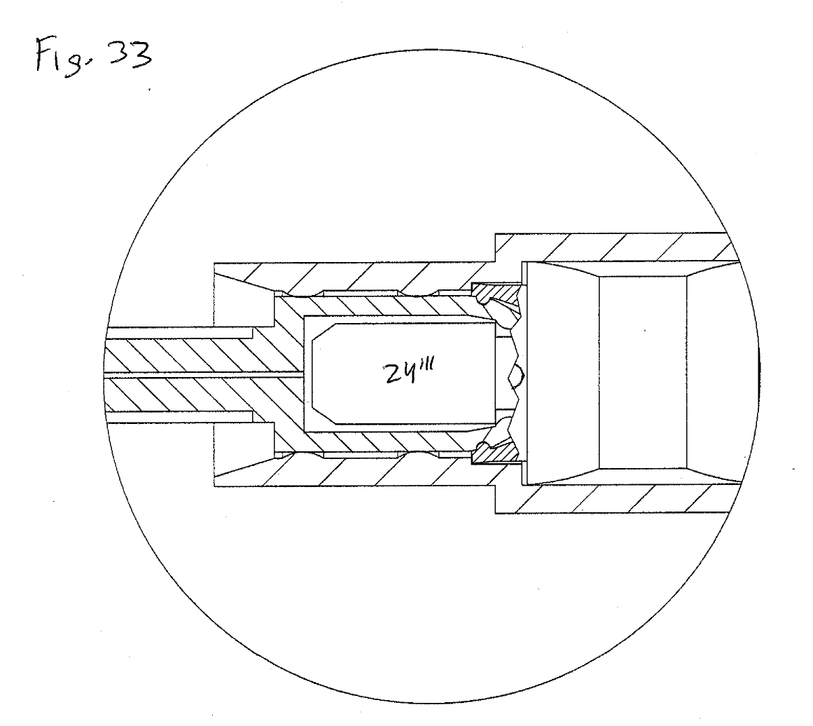

FIGS. 32-33 show a syringe according to another embodiment of the invention. In FIG. 32, the syringe is shown in an unlocked position. The syringe can assume this position prior to use. In this embodiment, the proximal end portion 24′″ of the plunger is a generally cylindrically shaped member with a tapered free end to allow for easy insertion into the opening 32′ of the needle support 30′. Furthermore, the portion 24′″ utilizes a distal shoulder 24″a which, when the plunger is in the locked position (i.e., the full injection position), is configured to engage with a circumferential inwardly projecting rib 34′a. However, as long as the plunger does not assume the substantially fully injection position (see FIG. 33), the portion 24′″ will not become locked to the needle support 30′. In order to lock the plunger to the needle support 30′, the embodiment shown in FIGS. 32-33 utilizes a sleeve-shaped plunger locking member which includes an inwardly facing circular locking projection LP is configured to lock with a circular locking recess LR arranged on an exterior surface of the needle support 30′. In order to ensure that the medicine is not prevented from exiting the syringe when the plunger is nearing the full injection position, the sleeve-shaped plunger locking member includes a plurality of through openings OP. These openings OP can be equally spaced and can be any number between, e.g., 1 and 12. Alternatively or in addition to the openings OP, the sleeve-shaped plunger locking member can include slots so as to form a plurality of spaced apart fingers each having a portion of the locking projection LP. As is evident from FIGS. 32 and 33, the plunger becomes locked to the needle support 30′ when the locking projection LP is received in the locking recess LR. FIG. 32 illustrates an intermediate position as the plunger moves towards the locked position. The syringe can assume this position during or after injection. FIG. 33 illustrates a locked position of the plunger and needle support 30′. As is evident from FIG. 33, the distal end of the needle support 30′ is forced to deflect inwardly as the sleeve-shaped plunger locking member moves over the distal end. Although not shown, the distal end of the needle support 30′ in the area of the locking recess LR can include slots so as to form a plurality of spaced apart fingers each having a portion of the locking recess LR. This would more easily allow the distal end to deflect inwardly and facilitate locking. FIG. 33 illustrates that when the plunger is locked to the needle support 30′, the syringe has substantially no dead space. Furthermore, because the proximal portion 24′″ is sized to substantially fill the space 32′ of the needle support 30′, the dead space within the space 32′ is substantially reduced. Although not shown, the plunger can cause the needle support 30′ (which is locked in a permanent or non-releasable manner thereto) to be retracted into the syringe barrel in the same manner as shown in FIG. 31.

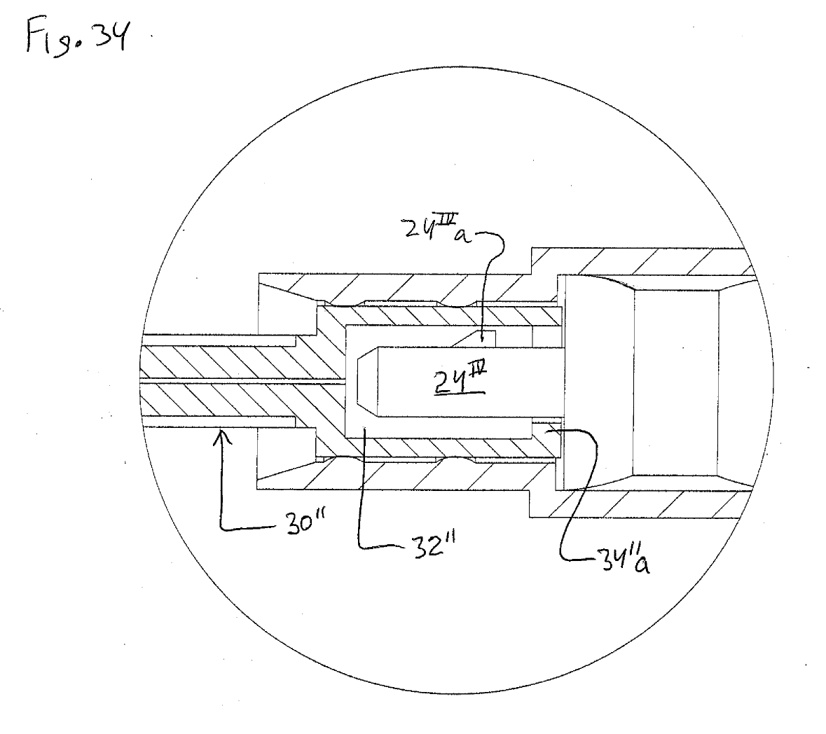

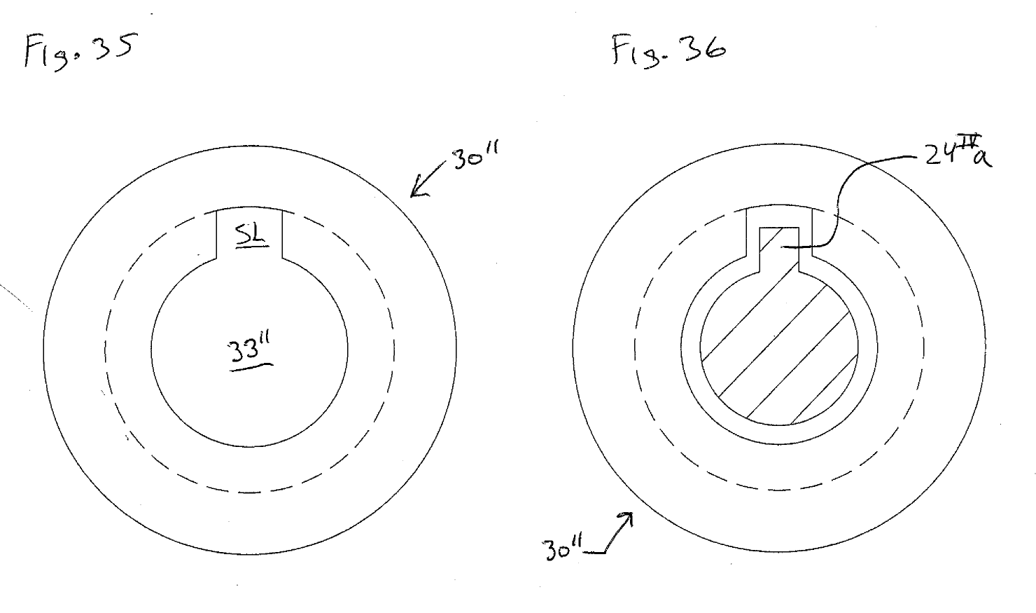

FIGS. 34-36 show a syringe according to another embodiment of the invention. In FIG. 34, the syringe is shown in an unlocked position. The syringe can assume this position prior to use and after use. In this embodiment, the proximal end portion 24^{IV} of the plunger is a generally cylindrically shaped member with a tapered free end to allow for easy insertion into the opening 32″ of the needle support 30″. Furthermore, the portion 24^{IV} utilizes a tapered projection 24^{IV}′a which, when the plunger is in the locked position (i.e., the plunger is moved to the full injection position and rotated by an angle in either direction that is sufficient to move the projection 24^{IV}a out of alignment with the slot SL formed in the needle support 30″), is configured to engage with a circumferential inwardly projecting rib 34″a. However, as long as the plunger is not rotated while in the substantially fully injection position (see FIG. 34), the portion 24^{IV} will not become locked to the needle support 30″. In order to lock the plunger to the needle support 30″, the embodiment shown in FIGS. 34-36 utilizes a tapered projection 24^{IV}a which is configured to lock with the circumferential inwardly projecting rib 34″a. As is evident from FIGS. 34-36, the plunger becomes locked to the needle support 30″ when the portion 24^{IV} is received in the space 32″ (after passing through opening 33″) and then rotated from the position shown in FIG. 36 to an angular position which positions the projection 24^{IV}aout of alignment (not shown) with the keying slot SL of the needle support 30″. The angle can be, e.g., as little as about 5 degrees or more, and in the embodiment shown in FIGS. 34-36 can be any value between about 5 degrees and about 355 degrees. Of course, this embodiment can also be modified to utilize two or more tapered projections utilize 24^{IV}a instead of just one.

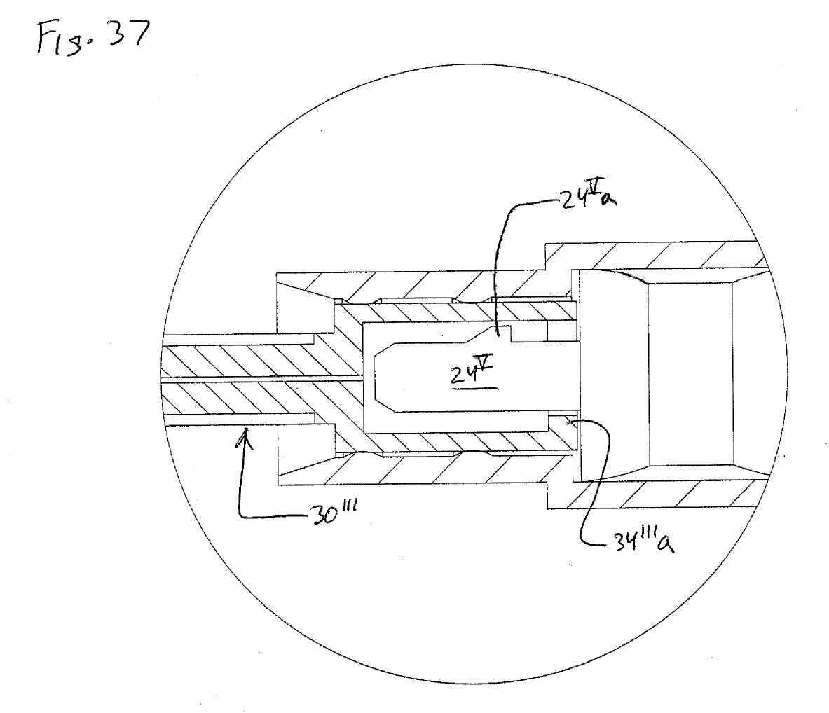

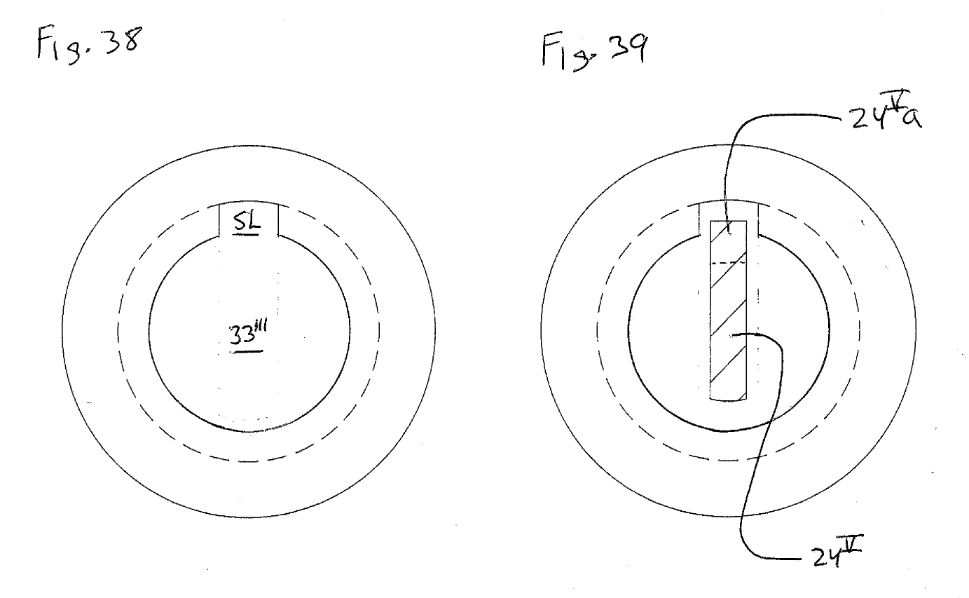

FIGS. 37-39 show a syringe according to another embodiment of the invention. In FIG. 37, the syringe is shown in an unlocked position. The syringe can assume this position prior to use and after use. In this embodiment, the proximal end portion 24^{V} of the plunger is a generally planar member (see FIG. 39) with a tapered free end to allow for easy insertion into the opening 32′″ of the needle support 30′″. Furthermore, the portion 24^{V} utilizes a tapered projection 24^{V}a which, when the plunger is in the locked position (i.e., the plunger is moved to the full injection position and rotated by an angle in either direction that is sufficient to move the projection 24^{V}a out of alignment with the slot SL formed in the needle support 30′″), is configured to engage with a circumferential inwardly projecting rib 34′″a. However, as long as the plunger is not rotated while in the substantially fully injection position (see FIG. 39), the portion 24^{V} will not become locked to the needle support 30″. In order to lock the plunger to the needle support 30′″, the embodiment shown in FIGS. 37-39 utilizes a tapered projection 24^{V}a which is configured to lock with the circumferential inwardly projecting rib 34′″a. As is evident from FIGS. 37-39, the plunger becomes locked to the needle support 30′″ when the portion 24^{V} is received in the space 32′″ (after passing through opening 33′″) and then rotated from the position shown in FIG. 39 to an angular position which positions the projection 24^{V}a out of alignment (not shown) with the keying slot SL of the needle support 30′″. The angle can be, e.g., as little as about 5 degrees or more, and in the embodiment shown in FIGS. 37-39 can be any value between about 5 degrees and about 355 degrees. Of course, this embodiment can also be modified to utilize two or more tapered projections utilize 24^{V}a instead of just one.

The syringe described above can also utilize one or more features disclosed in the prior art documents expressly incorporated by reference herein. Furthermore, one or more of the various parts of the syringe can preferably be made as one-piece structures by e.g., injection molding, when doing so reduces costs of manufacture. Non-limiting materials for most of the parts include synthetic resins such as those approved for syringes or other medical devices. Furthermore, the invention also contemplates that any or all disclosed features of one embodiment may be used on other disclosed embodiments, to the extent such modifications function for their intended purpose.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. An injection device comprising;
a barrel;
a plunger having a portion structured and arranged to move within the barrel;
a needle support; and
a locking arrangement that one of:
selectively locks a proximal end portion of the plunger to the needle support; and
automatically locks a proximal end portion of the plunger to an outer portion of the needle support.

2. The injection device of claim 1, wherein the injection device is at least one a syringe, a hypodermic syringe, a single-use syringe, and a retractable syringe.

3. The injection device of claim 1, wherein the barrel comprises a cylindrically shaped internal space structured and arranged to receive therein a medicine.

4. The injection device of claim 3, wherein the barrel further comprises a proximal opening within which the needle support is at least one of:
press fit; and
in sealing engagement therewith.

5. The injection device of claim 1, wherein the plunger comprises a piston arranged at a proximal area of the plunger and an actuating flange arranged at a distal area of the plunger.

6. The injection device of claim 5, wherein the plunger further comprises at least one of:
the piston being an elastomer or rubber piston;
a frangible section adapted to allow the plunger to be broken in two;
a stop flange adapted to limit forward movement of the plunger into the barrel; and
a stop projection adapted to limit rearward movement of the plunger out of the barrel.

7. The injection device of claim 1, wherein the barrel, the plunger, and the needle support each comprise a synthetic resin material.

8. The injection device of claim 1, wherein the needle support comprises a generally cylindrical distal portion and a needle projecting from a proximal end portion.

9. The injection device of claim 8, wherein the needle comprises at least one of metal and stainless steel.

10. The injection device of claim 1, wherein the barrel comprises at least one of:
a flange arranged at a distal end;
a reduced diameter portion arranged at a proximal end;
an internal taper arranged at a proximal end of the barrel adapted to facilitate insertion of the needle support; and
at least one internal sealing projection arranged within a proximal end opening of the barrel adapted to sealingly engage an outer surface of the needle support.

11. The injection device of claim 1, wherein the locking arrangement selectively locks a proximal end portion of the plunger to the needle support.

12. The injection device of claim 11, wherein the locking arrangement selectively locks the proximal end portion of the plunger to the needle support upon rotation of the plunger.

13. The injection device of claim 11, wherein the locking arrangement selectively locks the proximal end portion of the plunger to the needle support only upon rotation of the plunger.

14. The injection device of claim 11, wherein the locking arrangement selectively locks the proximal end portion of the plunger to the needle support upon rotation of the plunger to a predetermined position.

15. The injection device of claim 11, wherein the locking arrangement selectively locks the proximal end portion of the plunger to the needle support upon rotation of the plunger to a position approximately transverse to an initial unlocked position.

16. The injection device of claim 11, wherein the plunger is movable to a full insertion or to a full injection position without causing the proximal end portion of the plunger to become locked to the needle support.

17. The injection device of claim 11, wherein the plunger is movable to an injection position which minimizes a dead space without causing the proximal end portion of the plunger to become locked to the needle support.

18. The injection device of claim 11, wherein the plunger is movable to an injection position which causes contact between a piston of the plunger and an internal shoulder of the barrel without causing the proximal end portion of the plunger to become locked to the needle support.

19. The injection device of claim 11, wherein the plunger is movable to an injection position which causes contact between a portion of the plunger and an internal shoulder of the barrel without causing the proximal end portion of the plunger to become locked to the needle support.

20. The injection device of claim 11, wherein the proximal end portion of the plunger comprises at least one of:
a generally flat projecting portion with oppositely arranged projections adapted to engage, in the locked position, with internal projecting shoulders of the needle support;
a generally flat projecting portion with at least one projection adapted to engage, in the locked position, with at least one internal projecting shoulder of the needle support;
a projecting portion comprising opposite facing planar surfaces and oppositely arranged projections adapted to engage, in the locked position, with internal projecting shoulders of the needle support;
a projecting portion comprising opposite facing planar surfaces and at least one projection adapted to engage, in the locked position, with at least one internal projecting shoulder of the needle support;
a projecting portion comprising at least one tapered projection adapted to engage, in the locked position, with at least one internal projecting shoulder of the needle support;
a projecting portion comprising at least one fin-shaped projection adapted to engage, in the locked position, with at least one internal projecting shoulder of the needle support; and
a generally cylindrical projecting portion comprising at least one fin-shaped projection adapted to engage, in the locked position, with at least one internal projecting shoulder of the needle support.

21. The injection device of claim 1, wherein the locking arrangement automatically locks a proximal end portion of the plunger to an outer portion of the needle support.

22. The injection device of claim 21, wherein the locking arrangement automatically locks the proximal end portion of the plunger to the outer portion of the needle support when the plunger is moved to at least one of:
a full insertion position; and
a full injection position.

23. The injection device of claim 1, wherein a distal end portion of the needle support comprises at least one of;
a generally cylindrical opening sized to receive therein the proximal end portion of the plunger and at least one internal projecting shoulder adapted to engage, in the locked position, with at least one projection of the proximal end portion;
a generally cylindrical opening sized to receive therein the proximal end portion of the plunger and two oppositely arranged internal projecting shoulders adapted to engage, in the locked position, with at least one projection of the proximal end portion;
a generally cylindrical opening sized to receive therein the proximal end portion of the plunger and a generally rectangular-shaped distal entrance opening; and
a generally cylindrical opening sized to receive therein the proximal end portion of the plunger and a generally double D-shaped distal entrance opening.

24. A syringe comprising:
a barrel;
a plunger having a portion structured and arranged to move within the barrel;
a needle support; and
a locking arrangement that one of:
locks a proximal end portion of the plunger to the needle support when the plunger is rotated from an unlocked position to a locked position; and
locks the proximal end portion of the plunger to an outer portion of the needle support.

25. A syringe comprising:
a barrel comprising a main internal chamber for housing a medicine;
a plunger having a piston structured and arranged to move within the main internal chamber and thumb engaging flange arranged outside the barrel;
a needle support; and
a locking arrangement that is structured and arranged to one of:
lock a proximal end portion of the plunger to the needle support when the plunger is rotated from a first position to a second position while being in a full injection position; and
lock a proximal end portion of the plunger to an outer portion of the needle support after being moved to a full injection position,
wherein, when the plunger is in the full injection position, the syringe has essentially no dead space between the piston and an internal shoulder of the barrel.

26. A method of using the injection device of claim 1, the method comprising:
moving the plunger away from the needle support so as to cause a desired amount of medicine to enter into the barrel;
moving the plunger towards the needle support so as to cause medicine to exit the barrel through the needle support; and
locking the proximal end portion of the plunger to the needle support by rotating the plunger from a first position to a second position.

27. The method of claim 26, further comprising, after the locking, moving the plunger away from the needle support so as to cause the needle support to be fully positioned within the barrel.

28. A method of using the injection device of claim 1, the method comprising:
moving the plunger away from the needle support so as to cause a desired amount of medicine to enter into the barrel;
moving the plunger towards the needle support so as to cause medicine to exit the barrel through the needle support and so as to position the plunger at least one of:
at a full injection position; and
at a position substantially eliminating a dead space in the syringe;
again moving the plunger away from the needle support; and
locking the proximal end portion of the plunger to the needle support by rotating the plunger from a first position to a second position.

29. The method of claim 28, further comprising, after the locking, moving the plunger away from the needle support so as to cause the needle support to be fully positioned within the barrel.

30. A method of using the injection device of claim 1, the method comprising:
moving the plunger away from the needle support so as to cause a desired amount of medicine to enter into the barrel;
moving the plunger towards the needle support so as to cause medicine to exit the barrel through the needle support; and
locking the proximal end portion of the plunger to an outer portion of the needle support after the plunger is moved to a full injection position.

31. The method of claim 30, further comprising, after the locking, moving the plunger away from the needle support so as to cause the needle support to be fully positioned within the barrel.

32. A method of using the injection device of claim 1, the method comprising:
moving the plunger away from the needle support so as to cause a desired amount of medicine to enter into the barrel,
moving the plunger towards the needle support so as to cause medicine to exit the barrel through the needle support and so as to position the plunger at least one of:
at a full injection position; and
at a position substantially eliminating a dead space in the syringe;
again moving the plunger away from the needle support; and
locking the proximal end portion of the plunger to an outer portion of the needle support after the plunger is moved to a full injection position.

33. The method of claim 32, further comprising, after the locking, moving the plunger away from the needle support so as to cause the needle support to be fully positioned within the barrel.
